(19) United States
(12) Patent Application Publication
(10) Pub. No.: US 2026/0306656 A1
(43) Pub. Date: Oct. 8, 2026

(54) PROCESS FOR PURIFYING RED BLOOD CELL MEMBRANE FRAGMENTS

(71) Applicant: Ohio State Innovation Foundation, Columbus, OH (US)

(72) Inventors: Andre PALMER, Columbus, OH (US); Xiangming GU, Columbus, OH (US)

(21) Appl. No.: 19/480,696

(22) PCT Filed: May 1, 2024
(86) PCT No.: PCT/US2024/027243

§ 371 Date: Oct. 31, 2025

Related U.S. Application Data
(60) Provisional application No. 63/499,342, filed on May 1, 2023.

(51) Int. Cl. A61K 47/69; A61K 9/51; A61K 38/42; A61K 49/00; C12N 5/078; C12N 13/00

(52) U.S. Cl. CPC A61K 47/6901; A61K 9/5176; A61K 38/42; A61K 49/00; C12N 5/0641; C12N 13/00; A61K 2123/00; C12N 2510/00

(57) ABSTRACT

Disclosed herein are compositions comprising purified red blood cell membrane fragments (RBCMFs) as well as methods of making and using thereof.

### CROSS-REFERENCE TO RELATED APPLICATIONS

This application claims the benefit of, and priority to, U.S. Provisional Application No. 63/499,342, filed May 1, 2023, which is incorporated herein by reference in its entirety.

### STATEMENT REGARDING FEDERALLY SPONSORED RESEARCH OR DEVELOPMENT

This invention was made with government support under grant/contract nos. HL126945, HL138116, HL156526, HL158076, HL159862, HL162120, and EB021926 awarded by the National Institutes of Health and grant/contract no. W81XWH-18-1-0059 awarded by the Department of Defense. The government has certain rights in the invention.

### BACKGROUND

A challenge in the development of therapeutic nanoparticles (NPs) is their recognition by the immune system, and subsequent clearance from the circulation. The foreign surface properties of NPs lead to macrophage uptake and rapid clearance, which significantly reduces their circulatory half-life and therapeutic efficacy. These limitations are prevalent in many biomedical NP applications including drug delivery, diagnostics, and imaging. Therefore, various synthetic materials have been used to shield the NP surface to evade immune recognition. NP surface conjugation with poly (ethylene glycol) (PEG) is known to increase NP residence time, due to its hydrophilic surface properties. Unfortunately, there are concerns regarding the immunocompatibility of PEG. Some patients have preexisting PEG antibodies or develop PEG antibodies after repeated exposure to PEG. Polydopamine (PDA) surface coatings have also been studied and seem to impart anti-inflammatory properties by scavenging reactive oxygen species. However, there is a risk of forming non-covalently bound oligomers of dopamine, which do not attach to the NP surface and fail to confer benefits.

The use of red blood cell membrane fragments (RBCMFs) to surface-treat NPs has emerged as a robust and versatile approach for integrating natural and synthetic biomaterials to form NPs that evade immune recognition, since these coated NPs mimic the surface of red blood cells (RBCs) and mitigate macrophage uptake, while not requiring PEG or PDA surface conjugation. There is a distinction between the lysed, empty RBCMFs, and resealed RBCMFs, also known as resealed RBC ghosts (RBCGs). An advantage of RBCGs is that, when resealed via the addition of hypertonic solution, they are shown to maintain their outer membrane as ‘right side out’, rather than being turned inside out, the latter is linked to uptake by cellular recognition of phosphatidylserine that is traditionally present in the inner membrane leaflet. Purification of RBCMFs requires RBC lysis, and subsequent removal of cell-free hemoglobin (Hb) from the lysed RBCs, since cell-free Hb is cytotoxic. One can purify Hb from lysed RBCs at scale from lysed RBCs via tangential flow filtration (TFF). However, RBCMFs have generally been treated waste material. As such, the RBCMFs were not extensively characterized. Further, efforts were also not generally made to ensure the removal of the majority of cell-free Hb from the RBCMFs, since their focus was on purification of Hb, and not the RBCMFs. Other efforts in which isolation of RBCMFs did remove the majority of cell-free Hb used very low RBC volume fractions (<5%) and took hours to isolate the membranes, which is highly inefficient when considering the low concentration of recovered RBCMFs. This highlights the need for a scalable process to remove the majority of cell-free Hb from lysed RBCs at higher volume fractions, without unduly extending the time required to purify the RBCMFs.

Once RBCMFs are purified, there is also a need to develop a scalable platform for purification of RBCGs encapsulating a therapeutic agent, since the unencapsulated material must be removed from the RBCGs to prevent fast clearance of the therapeutic. Therefore, scalability is a key challenge towards creating a viable RBCG manufacturing platform, which can otherwise limit translation from bench-scale production to a commercial-scale production. Many processes for manufacturing RBCGs encapsulating a therapeutic agent are simply not scalable. Protocols utilizing microextrusion, microfluidics, or centrifugation as key steps in the production of therapeutic encapsulated RBCGs are difficult to scale.

Accordingly, improved methods of purifying RBCMFs and RBCGs are needed.

### SUMMARY

Provided herein are methods for forming a composition of purified red blood cell membrane fragments (RBCMFs). These methods can comprise: lysing a population of red blood cells (RBCs) to produce a composition comprising crude RBCMFs; and filtering the crude RBCMFs by microfiltration against a filtration membrane, thereby forming a retentate fraction comprising the purified RBCMFs and a permeate fraction comprising low molecular weight contaminants.

In some embodiments, the purified RBCMFs can comprise less than 1% by weight free hemoglobin and free heme, such as less than 0.5% by weight free hemoglobin and free heme, or less than 0.1% by weight free hemoglobin and free heme.

In some embodiments, the filtration membrane can be rated for a 0.65 μm molecular weight cut-off.

In some embodiments, filtering the crude RBCMFs by microfiltration comprises filtering for at least 3 diafiltrations cycles, such as at least 4 diafiltration cycles, at least 5 diafiltration cycles, at least 6 diafiltration cycles, at least 7 diafiltrations cycle, at least 8 diafiltration cycles, at least 9 diafiltrations cycle, or at least 10 diafiltration cycles.

In some embodiments, the microfiltration comprises tangential flow filtration.

In some embodiments, the method further comprises washing a population of red blood cells by microfiltration against a filtration membrane prior to the lysing step, thereby forming a retentate fraction comprising a population of washed red blood cells and a permeate fraction comprising cell-free hemoglobin and other low molecular weight contaminants.

In some embodiments, the washed red blood cells comprise less than 1% by weight free hemoglobin and free heme, such as less than 0.5% by weight free hemoglobin and free heme, or less than 0.1% by weight free hemoglobin and free heme.

In some embodiments, the filtration membrane used in the washing step is rated for a 0.65 μm molecular weight cut-off.

In some embodiments, washing the population of red blood cells by microfiltration comprises filtering for at least 3 diafiltrations cycles, such as at least 4 diafiltration cycles, at least 5 diafiltration cycles, at least 6 diafiltration cycles, at least 7 diafiltrations cycle, at least 8 diafiltration cycles, at least 9 diafiltrations cycle, or at least 10 diafiltration cycles.

In some embodiments, the microfiltration comprises tangential flow filtration.

In some embodiments, the method can further comprise modifying the size of the purified RBCMFs. Modifying the size of the purified RBCMFs can comprise, for example sonicating the purified RBCMFs, homogenizing a composition comprising the purified RBCMFs, shearing a composition comprising the purified RBCMFs, or extruding a composition comprising the purified RBCMFs to form a population of size-reduced RBCMFs.

In some embodiments, the population of purified size-reduced RBCMFs exhibit an average particle size of from 200 nm to 1 micron, as measured by dynamic light scattering (DLS).

In some embodiments, the population of purified size-reduced RBCMFs exhibit an average particle size of from 10 nm to 200 nm, as measured by dynamic light scattering (DLS).

In some embodiments, the method can further comprise encapsulating one or more active agent(s) in the purified RBCMFs or the size-reduced RBCMFs. In some embodiments, encapsulating the active agent(s) in the purified RBCMFs or the size-reduced RBCMFs can comprise contacting the purified RBCMFs or the size-reduced RBCMFs with the active agent(s) followed by ionic reversal to reseal the purified RBCMFs or the size-reduced RBCMFs. In some embodiments, encapsulating the active agent(s) in the purified RBCMFs or the size-reduced RBCMFs can comprise ionic reversal to reseal the purified RBCMFs or the size-reduced RBCMFs followed by electroporation to induce uptake of the active agent(s).

In some embodiments, the method can further comprise removing the unencapsulated active agent(s) from the active agent(s) encapsulated in the resealed RBCMFs or the resealed size-reduced RBCMFs via microfiltration or ultrafiltration.

In some embodiments, the filtration method to remove unencapsulated active agent can further comprise tangential flow filtration.

In some embodiments, the method can further comprise functionalizing a particle with the purified RBCMFs or the size-reduced RBCMFs.

In some embodiments, the method can further comprise conjugating one or more polymers to the purified RBCMFs or the size-reduced RBCMFs. In some embodiments, the polymer can comprise a hydrophilic polymer or a zwitterionic polymer. In certain embodiments, the polymer comprises a poly(alkylene oxide), such as polyethylene glycol (PEG).

The red blood cells can comprise human red blood cells or non-human red blood cells. In some embodiments, the red blood cells comprise red blood cells derived from stem cells, “recombinant red cells” cultured in bioreactors, or engineered red cells obtained from genetic engineering of stem cells.

Also provided are compositions comprising red blood cell ghosts (i.e. resealed RBCMFs) prepared by the methods described herein.

Also provided are compositions comprising a population of particles functionalized with RBCMFs prepared by the methods described herein.

Also provided are methods of using these compositions, for example, for drug delivery or diagnostics or combination of the two.

### DESCRIPTION OF DRAWINGS

FIG. 1 is a schematic of the RBCMF purification process. The left image shows a pictorial representation of the product at each step in the RBCMF purification process, first starting from intact RBCs, to RBCMFs, to sonicated RBCMFs. The right schematic shows the TFF process for RBC washing and subsequent RBCMF purification. The TFF system is similar for both the RBC washing and RBCMF purification processes, with only the type of pump differing between the two processes. A centrifugal pump was used for the RBC wash process to reduce hemolysis, while a peristaltic pump was used to purify RBCMFs from lysed RBCs.

FIGS. 2A-2E show the size analysis of RBCMFs. FIG. 2A shows DLS analysis of sonicated RBCMFs and 1.2 μm filtered unsonicated RBCMFs. FIG. 2B shows CC data comparing unsonicated RBCMFs and washed RBCs. FIGS. 2C, 2D, and 2E show TEM images illustrating the change in morphology going from RBCs, to RBCMFs, to sonicated RBCMFs. The magnifications used were 2250×, 11500×, and 28000× for FIG. 2C, FIG. 2D, and FIG. 2E respectively.

FIG. 3 shows cell-free Hb concentration during the TFF facilitated RBCMF purification process for both retentate (left) and permeate (right) samples. ANOVA testing revealed all differences to be significant at the p<0.05 level except for those marked with “Ψ”.

FIG. 4 shows the ζ potential of unsonicated and sonicated RBCMFs. The data includes both initially formed (unfrozen) RBCMFs; as well as the effects after freezing RBCMFs at −80° C. and subsequent thawing at various temperatures. ANOVA testing showed no significant differences between thawed groups for either sonicated or unsonicated RBCMFs.

FIGS. 5A-5C show the effects of the TFF washing process on select RBCMF properties. FIG. 5A indicates the time required to complete each diacycle. FIG. 5B shows the RBCMF concentration at each diacycle. FIG. 5C summarizes changes in average RBCMF particle diameter as measured by Coulter Counter. Notably, diacycles 1 and 2 have significantly different (p<0.05) mean RBCMF diameters than diacycles 6-10.

FIGS. 6A-6B show the oxygen binding properties of Hb-loaded RBCGs compared to human Hb and native RBCs. FIG. 6A represents the oxygen saturation of each material as a function of the partial pressure of oxygen (pO₂), while FIG. 6B represents the kinetics of oxygen offloading of each material as a function of time.

FIGS. 7A-7I show sonicated RBCMF particle coatings on zetag functionalized glass beads. FIGS. 7A-7C indicate the combined color channels, GFP channel, and mCherry channel for the RBCMF coating onto the non-fluorescent zetag-treated bead, respectively. FIGS. 7D-7F indicate the same color channels as FIGS. 7A-7C, however with the GFP-tagged zetag. Red fluorescence is indicative of WGA, which binds to membrane glycoproteins present on RBCMFs, and is indicative of a surface coating on the bead. FIGS. 7G-7I indicate a control where no RBCMFs were applied to a bead functionalized with a non-GFP zetag.

FIG. 8 illustrates the RBC wash and RBCMF purification process. The TFF system is similar for both the RBC washing and RBCMF purification processes, with only the type of pump and wash solution differing between the two processes. A centrifugal pump was used for the RBC wash process to reduce hemolysis, while a peristaltic pump was used to purify RBCMFs from lysed RBCs.

FIG. 9 illustrates the RBCMF resealing process to encapsulate Hb to yield washed Hb-RBCGs.

FIG. 10 shows TEM images for expired RBCs (panel A), RBCMFs (panel B), and Hb-RBCGs (panel C).

FIGS. 11A-11B show cell-free Hb concentration during the TFF facilitated RBCMF purification process for both retentate (FIG. 11A) and permeate (FIG. 11B) samples. ANOVA testing revealed all differences to be significant at the p<0.05 level except for those marked with “Ψ”.

FIGS. 12A-12C show the effects of the TFF Washing Process on Diacycle Time and RBCMF Properties. FIG. 12A indicates the time required to complete each diacycle. FIG. 12B shows the RBCMF concentration at each diacycle. FIG. 12C summarizes changes in average RBCMF particle diameter as measured by CC. Notably, diacycles 1 and 2 have significantly different (p<0.05) mean RBCMF diameters than diacycles 6-10.

FIG. 13 shows the ζ potential of RBCMFs. The data includes both freshly prepared (unfrozen) RBCMFs; as well as the effects after freezing RBCMFs at −80° C. and subsequent thawing at various temperatures. ANOVA testing showed no significant differences between thawed RBCMF groups.

FIG. 14 shows the effect of different wash methods (centrifugation versus TFF) for purifying Hb-RBCGs on protein (Hb) encapsulation inside Hb-RBCGs. Hb-RBCGs washed via centrifugation had significantly lower Hb encapsulation efficiency compared to Hb-RBCGs washed via TFF (*p<0.05). The Hb encapsulation efficiency was normalized by the Hb-RBCG volume fraction to account for batch-to-batch variation in the precursor RBCMFs.

FIGS. 15A-15B show the oxygen binding properties of Hb-RBCGs compared to cell-free Hb and fresh RBCs. Oxygen equilibrium curve (FIG. 15A) and oxygen offloading kinetics (FIG. 15B). Representative curves were selected for each sample from a total of n=6 different preparations for the OEC curves, and n=5 different preparations for the oxygen offloading kinetics.

FIG. 16 shows RBCG morphology at various stages in the RBCG production process. (panel A) RBCs before washing. (panel B) RBCs after washing. (panel C) RBCMFs. (panel D) Hb-RBCGs-C highlighting their tendency to aggregate into extremely large particles. (panel E) Empty RBCGs, showing similar patterns of aggregation to a lesser extent. (panel F) Hb-RBCGs-TFF do not show signs of the aggregation observed in panel D and panel E.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

As used in the description and the appended claims, the singular forms “a,” “an,” and “the” include plural referents unless the context clearly dictates otherwise.

As used herein, the terms “optional” or “optionally” mean that the subsequently described event or circumstance may or may not occur and that the description includes instances where said event or circumstance occurs and instances where it does not.

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this invention belongs. In this specification and in the claims which follow, reference will be made to a number of terms that shall be defined herein.

For the terms “for example” and “such as,” and grammatical equivalences thereof, the phrase “and without limitation” is understood to follow unless explicitly stated otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

It will be understood that, although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of example embodiments.

As used herein the term “substantially” means that the subsequently described event or circumstance completely occurs or that the subsequently described event or circumstance generally, typically, or approximately occurs.

As used herein, the term “substantially,” in, for example, the context “substantially identical” or “substantially similar” refers to a method or a system, or a component that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

It is understood that in aspects disclosed herein, the terms “diacycle” and “diafiltration cycles” are used interchangeably.

As used herein, the term “tangential-flow filtration” refers to a process in which the fluid mixture containing the components to be separated by filtration is recirculated at velocities tangential to the plane of the filtration membrane to reduce fouling of the filter. In such filtrations a pressure differential is applied along the length of the filtration membrane to cause the fluid and filterable solutes to flow through the membrane (i.e., filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of aspects described in the specification.

### Methods

Provided herein are methods for forming a composition of purified red blood cell membrane fragments (RBCMFs). These methods can comprise: lysing a population of red blood cells (RBCs) to produce a composition comprising crude RBCMFs; and filtering the crude RBCMFs by microfiltration against a filtration membrane, thereby forming a retentate fraction comprising the purified RBCMFs and a permeate fraction comprising low molecular weight contaminants.

The red blood cells can comprise human red blood cells or non-human red blood cells. In some embodiments, the red blood cells can comprise human red blood cells isolated from whole blood. In other embodiments, the red blood cells comprise red blood cells derived from stem cells, “recombinant red cells” cultured in bioreactors, or engineered red cells obtained from genetic engineering of stem cells.

In some embodiments, the purified RBCMFs can comprise less than 1% by weight (e.g., less than 0.75% by weight, less than 0.5% by weight, less than 0.4% by weight, less than 0.3% by weight, less than 0.25% by weight, less than 0.2% by weight, less than 0.1% by weight, less than 0.05% by weight, or less than 0.01% by weight) free hemoglobin and free heme. In some embodiments, the purified RBCMFs can comprise no detectable free hemoglobin and free heme.

Lysing the population of RBCs can comprise any suitable method for inducing lysis of cells. In some embodiments, this can comprise contacting the population of RBCs with a lysing agent, such as water or a buffer solution having an appropriate ionic strength to induce lysis of the RBCs. Suitable lysing agents are known in the art, including commercially available RBC lysis buffers, which are ammonium chloride-based buffer systems designed for the preferential lysis of red blood cells from whole blood.

Filtration can be performed using any suitable membrane having an appropriate pore size to retain the RBCMFs while allowing impurities to pass to the permeate size of the membrane. In some examples, the filtration membrane can be rated as having a pore size of 0.1 μm, 0.2 μm, 0.45 μm, 0.65 μm, 0.8 μm, 1 μm, or 1.2 μm. In certain examples, the filtration membrane can be rated as having a pore size of 0.65 μm. In some embodiments, the microfiltration comprises tangential flow filtration.

In some embodiments, filtering the crude RBCMFs by microfiltration comprises filtering for at least 3 diafiltrations cycles, such as at least 4 diafiltration cycles, at least 5 diafiltration cycles, at least 6 diafiltration cycles, at least 7 diafiltrations cycle, at least 8 diafiltration cycles, at least 9 diafiltrations cycle, or at least 10 diafiltration cycles. In some embodiments, filtering the crude RBCMFs by microfiltration comprises filtering for 25 diafiltration cycles or less (e.g., 20 diafiltration cycles or less, 15 diafiltration cycles or less, or 10 diafiltration cycles or less).

In some embodiments, the method further comprises washing a population of red blood cells by microfiltration against a filtration membrane prior to the lysing step, thereby forming a retentate fraction comprising a population of washed red blood cells and a permeate fraction comprising cell-free hemoglobin and other low molecular weight contaminants.

In some embodiments, the washed red blood cells can comprise less than 1% by weight (e.g., less than 0.75% by weight, less than 0.5% by weight, less than 0.4% by weight, less than 0.3% by weight, less than 0.25% by weight, less than 0.2% by weight, less than 0.1% by weight, less than 0.05% by weight, or less than 0.01% by weight) free hemoglobin and free heme. In some embodiments, the washed red blood cells can comprise no detectable free hemoglobin and free heme.

Filtration can be performed using any suitable membrane having an appropriate pore size to retain the red blood cells while allowing impurities to pass to the permeate size of the membrane. In some examples, the filtration membrane can be rated as having a pore size of 0.1 μm, 0.2 μm, 0.45 μm, 0.65 μm, 0.8 μm, 1 μm, or 1.2 μm. In certain examples, the filtration membrane can be rated as having a pore size of 0.65 μm. In some embodiments, the microfiltration comprises tangential flow filtration.

In some embodiments, filtering the red blood cells by microfiltration comprises filtering for at least 3 diafiltrations cycles, such as at least 4 diafiltration cycles, at least 5 diafiltration cycles, at least 6 diafiltration cycles, at least 7 diafiltrations cycle, at least 8 diafiltration cycles, at least 9 diafiltrations cycle, or at least 10 diafiltration cycles. In some embodiments, filtering the red blood cells by microfiltration comprises filtering for 25 diafiltration cycles or less (e.g., 20 diafiltration cycles or less, 15 diafiltration cycles or less, or 10 diafiltration cycles or less).

In some embodiments, the method can further comprise modifying the size of the purified RBCMFs. Modifying the size of the purified RBCMFs can comprise, for example sonicating the purified RBCMFs, homogenizing a composition comprising the purified RBCMFs, shearing a composition comprising the purified RBCMFs, or extruding a composition comprising the purified RBCMFs to form a population of size-reduced RBCMFs.

In some embodiments, the population of purified size-reduced RBCMFs exhibit an average particle size of at least 10 nm (e.g., at least 20 nm, at least 25 nm, at least 30 nm, at least 40 nm, at least 50 nm, at least 60 nm, at least 70 nm, at least 75 nm, at least 80 nm, at least 90 nm, at least 100 nm, at least 110 nm, at least 120 nm, at least 125 nm, at least 130 nm, at least 140 nm, at least 150 nm, at least 160 nm, at least 170 nm, at least 175 nm, at least 180 nm, or at least 190 nm), as measured by dynamic light scattering (DLS). In some embodiments, the population of purified size-reduced RBCMFs exhibit an average particle size of 200 nm or less (e.g., 190 nm or less, 180 nm or less, 175 nm or less, 170 nm or less, 160 nm or less, 150 nm or less, 140 nm or less, 130 nm or less, 125 nm or less, 120 nm or less, 110 nm or less, 100 nm or less, 90 nm or less, 80 nm or less, 75 nm or less, 70 nm or less, 60 nm or less, 50 nm or less, 40 nm or less, 30 nm or less, 25 nm or less, or 20 nm or less), as measured by dynamic light scattering (DLS).

The population of purified size-reduced RBCMFs can exhibit an average particle size ranging from any of the minimum values described above to any of the maximum values described above. For example, in some embodiments, the population of purified size-reduced RBCMFs exhibit an average particle size of from 10 nm to 200 nm, as measured by dynamic light scattering (DLS).

In some embodiments, the method can further comprise encapsulating one or more active agent(s) in the purified RBCMFs or the size-reduced RBCMFs. In some embodiments, encapsulating the active agent(s) in the purified RBCMFs or the size-reduced RBCMFs can comprise contacting the purified RBCMFs or the size-reduced RBCMFs with the active agent(s) followed by ionic reversal to reseal the purified RBCMFs or the size-reduced RBCMFs. In some embodiments, encapsulating the active agent(s) in the purified RBCMFs or the size-reduced RBCMFs can comprise ionic reversal to reseal the purified RBCMFs or the size-reduced RBCMFs followed by electroporation to induce uptake of the active agent(s).

In some embodiments, the method can further comprise removing the unencapsulated active agent(s) from the active agent(s) encapsulated in the resealed RBCMFs or the resealed size-reduced RBCMFs via microfiltration or ultrafiltration. For example, the method can comprise filtering the resealed RBCMFs or the resealed size-reduced RBCMFs against a filtration membrane, thereby forming a retentate fraction comprising the purified resealed RBCMFs or the resealed size-reduced RBCMFs and a permeate fraction comprising any unencapsulated active agent(s). In some embodiments, the filtration method to remove unencapsulated active agent can further comprise tangential flow filtration.

In the methods described herein, the active agent can comprise any suitable active agent. Examples of active agents include, for example, therapeutic agents (e.g. anti-cancer agents), diagnostic agents (e.g. contrast agents; radionuclides; and fluorescent, luminescent, and magnetic moieties), prophylactic agents (e.g. vaccines), and/or nutraceutical agents (e.g. vitamins, minerals, etc.) may be delivered by the disclosed nanoparticles. Exemplary agents to be delivered in accordance with the present invention include, but are not limited to, small molecules (e.g. cytotoxic agents), nucleic acids (e.g., siRNA, RNAi, and microRNA agents), proteins (e.g. antibodies), peptides, lipids, carbohydrates, hormones, metals, radioactive elements and compounds, drugs, vaccines, immunological agents, etc., and/or combinations thereof. In some embodiments, the agent to be delivered is an agent useful in the treatment of cancer (e.g., breast, lung, or prostate cancer).

The active agent or drug may be a therapeutic agent such as mTor inhibitors (e.g., sirolimus, temsirolimus, or everolimus), vinca alkaloids (e.g. vinorelbine or vincristine), a diterpene derivative, a taxane (e.g. paclitaxel or its derivatives such as DHA-paclitaxel or PG-paxlitaxelor, or docetaxel), a boronate ester or peptide boronic acid compound (e.g. bortezomib), a cardiovascular agent (e.g. a diuretic, a vasodilator, angiotensin converting enzyme, a beta blocker, an aldosterone antagonist, or a blood thinner), a corticosteroid (e.g. budensonide, fluocinonide, triamcinolone, mometasone, amcinonide, halcinonide, ciclesonide, beclomethansone), an antimetabolite or antifolate agent (e.g. methotrexate), a chemotherapeutic agent (e.g. epothilone B), a nitrogen mustard agent (e.g. bendamustine), or the active agent or drug may be an siRNA.

Example therapeutic agents include chemotherapeutic agents such as doxorubicin (adriamycin), gemcitabine (gemzar), daunorubicin, procarbazine, mitomycin, cytarabine, etoposide, methotrexate, 5-fluorouracil (5-FU), vinca alkaloids such as vinblastine, vinoelbine, vindesine, or vincristine; bleomycin, taxanes such as paclitaxel (taxol) or docetaxel (taxotere), mTOR inhibitors such as sirolimus, temsirolimus, or everolimus, aldesleukin, asparaginase, boronate esters or peptide boronic acid compounds such as bortezomib, busulfan, carboplatin, cladribine, camptothecin, CPT-11, 10-hydroxy-7-ethylcamptothecin (SN38), dacarbazine, S-I capecitabine, ftorafur, 5′deoxyflurouridine, UFT, eniluracil, deoxycytidine, 5-azacytosine, 5-azadeoxycytosine, allopurinol, 2-chloroadenosine, trimetrexate, aminopterin, methylene-10-deazaaminopterin (MDAM), oxaplatin, picoplatin, tetraplatin, satraplatin, platinum-DACH, ormaplatin, CI-973, JM-216, epirubicin, etoposide phosphate, 9-aminocamptothecin, 10,11-methylenedioxycamptothecin, karenitecin, 9-nitrocamptothecin, TAS 103, L-phenylalanine mustard, ifosphamidemefosphamide, perfosfamide, trophosphamide carmustine, semustine, bendamustine, epothilones A-E, tomudex, 6-mercaptopurine, 6-thioguanine, amsacrine, karenitecin, acyclovir, valacyclovir, ganciclovir, amantadine, rimantadine, lamivudine, zidovudine, bevacizumab, trastuzumab, rituximab, budesonide, and combinations thereof, or the therapeutic agent may be an siRNA.

Non-limiting examples of potentially suitable active agents include anti-cancer agents, including, for example, docetaxel, mitoxantrone, and mitoxantrone hydrochloride. In another embodiment, the payload may be an anti-cancer drug such as 20-epi-1, 25 dihydroxyvitamin D3, 4-ipomeanol, 5-ethynyluracil, 9-dihydrotaxol, abiraterone, acivicin, aclarubicin, acodazole hydrochloride, acronine, acylfiilvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizdng morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisazuidinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caraceraide, carbetimer, carboplatin, carboxamide-amino-triazole, carboxyamidotriazole, carest M3, carmustine, earn 700, cartilage derived inhibitor, carubicin hydrochloride, carzelesin, casein kinase inhibitors, castanosperrnine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cyclosporine, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethyhiorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocannycin SA, ebselen, ecomustine, edatrexate, edelfosine, edrecolomab, eflomithine, eflomithine hydrochloride, elemene, elsarnitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, flurocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ihnofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatm, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide, lobaplatin, lombricine, lometrexol, lometrexol sodium, lomustine, lonidamine, losoxantrone, losoxantrone hydrochloride, lovastatin, loxoribine, lurtotecan, lutetium texaphyrin lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C uihibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazorurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinarnide, RNAi, rogletimide, rohitukine, romurtide, roquinimex, rubiginone B1, ruboxyl, safingol, safingol hydrochloride, saintopin, sarcnu, sarcophytol A, sargramostim, SDI1 mimetics, semustine, senescence derived inhibitor 1, sense oligonucleotides, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosafe sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine or vinorelbine tartrate, vinrosidine sulfate, vinxaltine, vinzolidine sulfate, vitaxin, vorozole, zanoterone, zeniplatin, zilascorb, zinostatin, zinostatin stimalamer, and zorubicin hydrochloride.

In some embodiments, the method can further comprise functionalizing a particle with the purified RBCMFs or the size-reduced RBCMFs.

In certain embodiments, the method can further comprise conjugating one or more polymers to the purified RBCMFs or the size-reduced RBCMFs. The polymer can comprise any suitable polymer. In some examples, the polymer can comprise a hydrophilic polymer or a zwitterionic polymer.

Examples of suitable polymers can include poly(alkylene oxides), such as polyethylene glycol (PEG), polyesters, including polymers and copolymers comprising lactic acid and/or glycolic acid units, such as polylactic acid, polyglycolic acid, poly(lactic)-co-poly(glycolic) acid, poly(lactic acid-co-glycolic acid), and poly(lactide-co-glycolide), polyhydroxyacids, and/or polyanhydrides, poly(ortho ester), PEGylated poly(ortho ester), polylysine, PEGylated polylysine, poly(ethylene imine), PEGylated poly(ethylene imine), poly(L-lactide-co-L-lysine), poly(serine ester), poly(4-hydroxy-L-proline ester), poly[α-(4-aminobutyl)-L-glycolic acid], polyesters bearing cationic side chains, such as poly(L-lactide-co-L-lysine), poly(serine ester), poly(4-hydroxy-L-proline ester), acrylic polymers, such as acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), methacrylic acid alkylamide copolymer, poly(methyl methacrylate), poly(methacrylic acid polyacrylamide, amino alkyl methacrylate copolymer, glycidyl methacrylate copolymers, polycyanoacrylates, copolymers thereof, and blends thereof.

Particles Functionalized with RBCMFs

Also provided are particles functionalized with RBCMFs prepared by the methods described herein.

Example particles include polymeric particles, lipid particles, solid lipid particles, inorganic particles, or combinations thereof (e.g., lipid stabilized polymeric particles). In some embodiments, the particles are polymeric particles or contain a polymeric matrix.

The particles can contain any of the polymers described herein or derivatives or copolymers thereof. The particles generally contain one or more biocompatible polymers. The polymers can be biodegradable polymers. The polymers can be hydrophobic polymers, hydrophilic polymers, or amphiphilic polymers.

The size of the particles can be adjusted for the intended application. The particles can be nanoparticles or microparticles. The particle can have a diameter of about 10 nm to about 10 microns, about 10 nm to about 1 micron, about 10 nm to about 500 nm, about 20 nm to about 500 nm, or about 25 nm to about 250 nm. In some embodiments the particle is a nanoparticle having a diameter from about 25 nm to about 250 nm. It is understood by those in the art that a plurality of particles will have a range of sizes and the diameter is understood to be the median diameter of the particle size distribution.

In various embodiments, a particle may be a nanoparticle, i.e., the particle has a characteristic dimension of less than about 1 micrometer, where the characteristic dimension of a particle is the diameter of a perfect sphere having the same volume as the particle. The plurality of particles can be characterized by an average diameter (e.g., the average diameter for the plurality of particles). In some embodiments, the diameter of the particles may have a Gaussian-type distribution. In some embodiments, the plurality of particles have an average diameter of less than about 300 nm, less than about 250 nm, less than about 200 nm, less than about 150 nm, less than about 100 nm, less than about 50 nm, less than about 30 nm, less than about 10 nm, less than about 3 nm, or less than about 1 nm. In some embodiments, the particles have an average diameter of at least about 5 nm, at least about 10 nm, at least about 30 nm, at least about 50 nm, at least about 100 nm, at least about 150 nm, or greater. In certain embodiments, the plurality of the particles have an average diameter of about 10 nm, about 25 nm, about 50 nm, about 100 nm, about 150 nm, about 200 nm, about 250 nm, about 300 nm, about 500 nm, or the like. In some embodiments, the plurality of particles have an average diameter between about 10 nm and about 500 nm, between about 50 nm and about 400 nm, between about 100 nm and about 300 nm, between about 150 nm and about 250 nm, between about 175 nm and about 225 nm, or the like. In some embodiments, the plurality of particles have an average diameter between about 10 nm and about 500 nm, between about 20 nm and about 400 nm, between about 30 nm and about 300 nm, between about 40 nm and about 200 nm, between about 50 nm and about 175 nm, between about 60 nm and about 150 nm, between about 70 nm and about 130 nm, or the like. For example, the average diameter can be between about 70 nm and 130 nm. In some embodiments, the plurality of particles have an average diameter between about 20 nm and about 220 nm, between about 30 nm and about 200 nm, between about 40 nm and about 180 nm, between about 50 nm and about 170 nm, between about 60 nm and about 150 nm, or between about 70 nm and about 130 nm. In one embodiment, the particles have a size of 40 to 120 nm with a zeta potential close to 0 mV at low to zero ionic strengths (1 to 10 mM), with zeta potential values between +5 to −5 mV, and a zero/neutral or a small −ve surface charge.

Example particles include microparticle and nanoparticle compositions conventionally utilized to deliver active agent cargos, including polymeric microparticles and nanoparticles loaded with active agents, lipid nanoparticles, liposomes, vesicles, and other drug delivery microparticles and nanoparticles. The surface of these particles can be decorated with RBCMFs prepared by the methods described herein. This can reduce clearance of the microparticles or nanoparticles by the immune system of a patient upon administration.

The RBCMFs prepared by the methods described herein can be associated with the surface of the microparticles or nanoparticles either non-covalently (e.g., by electrostatic and/or Van der Waals interactions with the surface of the microparticles or nanoparticles) or covalently (e.g., by conjugation of the RBCMFs or the particle surface or by conjugation of the RBCMFs to a component used to form the microparticles or nanoparticles).

By way of example, provided herein are polymeric particles (e.g., microparticles or nanoparticles) that include a matrix of polymers, a therapeutic agent, and RBCMFs prepared by the methods described associated with the surface of the microparticles or nanoparticles.

The polymeric particles can comprise one or more polymers or copolymers. Various molecular weights of polymers are contemplated herein, for example, the weight of a polymer may influence particle degradation rate, solubility, water uptake, and drug release kinetics. The molecular weight of the polymer can be adjusted such that the particle biodegrades in the subject being treated within a reasonable period of time (ranging from a few hours to 1-2 weeks, 3-4 weeks, 5-6 weeks, 7-8 weeks, etc.) For example, a disclosed particle may comprise a copolymer of PLA and PEG or PLGA and PEG, wherein the PLA or PLGA portion may have a number average molecule weight of about 30 kDa to about 90 kDa or about 40 kDa to about 90 kDa, and the PEG portion may have a molecular weight of about 4 kDa to about 6 kDa. In an exemplary embodiment, the PLA or the PLGA portion may have a number average molecule weight of 30 kDa, 50 kDa, 65 kDa, or 80 kDa. The PEG portion may have a molecular weight of about 5 kDa, about 6, 7, 8, or 9 kDa, or about 10 kDa.

The particles may include one or more polymers, e.g. a first polymer that may be a co-polymer, e.g. a diblock co-polymer, and optionally a polymer that may be for example a homopolymer. In some embodiments, the particles can include a matrix of polymers. In some embodiments, the active agent can be associated with the surface of, encapsulated within, surrounded by, and/or dispersed throughout a polymeric matrix.

Disclosed particles can include copolymers, which, in some embodiments, describe two or more polymers (such as those described herein) that have been associated with each other, usually by covalent bonding of the two or more polymers together. Thus, a copolymer may comprise a first polymer and a second polymer, which have been conjugated together to form a block copolymer where the first polymer can be a first block of the block copolymer and the second polymer can be a second block of the block copolymer. Of course, those of ordinary skill in the art will understand that a block copolymer may, in some cases, contain multiple blocks of polymer, and that a “block copolymer,” as used herein, is not limited to only block copolymers having only a single first block and a single second block. For instance, a block copolymer may comprise a first block comprising a first polymer, a second block comprising a second polymer, and a third block comprising a third polymer or the first polymer, etc. In some cases, block copolymers can contain any number of first blocks of a first polymer and second blocks of a second polymer (and in certain cases, third blocks, fourth blocks, etc.). In addition, it should be noted that block copolymers can also be formed, in some instances, from other block copolymers. For example, a first block copolymer may be conjugated to another polymer (which may be a homopolymer, a biopolymer, another block copolymer, etc.), to form a new block copolymer containing multiple types of blocks, and/or to other moieties (e.g., to non-polymeric moieties).

In some embodiments, a contemplated copolymer (e.g., block copolymer) can be amphiphilic, i.e., having a hydrophilic portion and a hydrophobic portion, or a relatively hydrophilic portion and a relatively hydrophobic portion. A hydrophilic polymer can be one generally that attracts water and a hydrophobic polymer can be one that generally repels water. A hydrophilic or a hydrophobic polymer can be identified, for example, by preparing a sample of the polymer and measuring its contact angle with water (typically, the polymer will have a contact angle of less than 60°, while a hydrophobic polymer will have a contact angle of greater than about 60°). In some cases, the hydrophilicity of two or more polymers may be measured relative to each other, i.e., a first polymer may be more hydrophilic than a second polymer. For instance, the first polymer may have a smaller contact angle than the second polymer.

In one set of embodiments, a high molecular weight polymer (e.g., copolymer, e.g., block copolymer) contemplated herein includes a biocompatible polymer, i.e., the polymer that does not typically induce an adverse response when inserted or injected into a living subject, for example, without significant inflammation and/or acute rejection of the polymer by the immune system, for instance, via a T-cell response. Accordingly, the therapeutic particles contemplated herein can be non-immunogenic. The term non-immunogenic as used herein refers to endogenous growth factor in its native state which normally elicits no, or only minimal levels of, circulating antibodies, T-cells, or reactive immune cells, and which normally does not elicit in the individual an immune response against itself.

Biocompatibility typically refers to the acute rejection of material by at least a portion of the immune system, i.e., a nonbiocompatible material implanted into a subject provokes an immune response in the subject that can be severe enough such that the rejection of the material by the immune system cannot be adequately controlled, and often is of a degree such that the material must be removed from the subject. One simple test to determine biocompatibility can be to expose a polymer to cells in vitro; biocompatible polymers are polymers that typically will not result in significant cell death at moderate concentrations, e.g., at concentrations of 50 micrograms/10⁶ cells. For instance, a biocompatible polymer may cause less than about 20% cell death when exposed to cells such as fibroblasts or epithelial cells, even if phagocytosed or otherwise uptaken by such cells. Non-limiting examples of biocompatible polymers that may be useful in various embodiments of the present invention include polydioxanone (PDO), polyhydroxyalkanoate, polyhydroxybutyrate, poly(glycerol sebacate), polyglycolide, polylactide, PLGA, PLA, polycaprolactone, or copolymers or derivatives including these and/or other polymers.

In certain embodiments, contemplated biocompatible polymers may be biodegradable, i.e., the polymer is able to degrade, chemically and/or biologically, within a physiological environment, such as within the body. As used herein, “biodegradable” polymers are those that, when introduced into cells, are broken down by the cellular machinery (biologically degradable) and/or by a chemical process, such as hydrolysis, (chemically degradable) into components that the cells can either reuse or dispose of without significant toxic effect on the cells. In one embodiment, the biodegradable polymer and their degradation byproducts can be biocompatible.

For instance, a contemplated polymer may be one that hydrolyzes spontaneously upon exposure to water (e.g., within a subject), the polymer may degrade upon exposure to heat (e.g., at temperatures of about 37° C.). Degradation of a polymer may occur at varying rates, depending on the polymer or copolymer used. For example, the half-life of the polymer (the time at which 50% of the polymer can be degraded into monomers and/or other nonpolymeric moieties) may be on the order of days, weeks, months, or years, depending on the polymer. The polymers may be biologically degraded, e.g., by enzymatic activity or cellular machinery, in some cases, for example, through exposure to a lysozyme (e.g., having relatively low pH). In some cases, the polymers may be broken down into monomers and/or other nonpolymeric moieties that cells can either reuse or dispose of without significant toxic effect on the cells (for example, polylactide may be hydrolyzed to form lactic acid, polyglycolide may be hydrolyzed to form glycolic acid, etc.).

In some embodiments, polymers comprise one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(F-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker.

The particles may contain one or more hydrophilic polymers. Hydrophilic polymers include cellulosic polymers such as starch and polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol); polyoxazoline; and copolymers thereof.

The particles may contain one or more hydrophobic polymers. Examples of suitable hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof.

In certain embodiments, the hydrophobic polymer is an aliphatic polyester. In some embodiments, the hydrophobic polymer is poly(lactic acid), poly(glycolic acid), or poly(lactic acid-co-glycolic acid).

The particles can contain one or more biodegradable polymers. Biodegradable polymers can include polymers that are insoluble or sparingly soluble in water that are converted chemically or enzymatically in the body into water-soluble materials.

Biodegradable polymers can include soluble polymers crosslinked by hydolyzable cross-linking groups to render the crosslinked polymer insoluble or sparingly soluble in water.

Biodegradable polymers in the particle can include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof alkyl cellulose such as methyl cellulose and ethyl cellulose, hydroxyalkyl celluloses such as hydroxypropyl cellulose, hydroxy-propyl methyl cellulose, and hydroxybutyl methyl cellulose, cellulose ethers, cellulose esters, nitro celluloses, cellulose acetate, cellulose propionate, cellulose acetate butyrate, cellulose acetate phthalate, carboxylethyl cellulose, cellulose triacetate, cellulose sulphate sodium salt, polymers of acrylic and methacrylic esters such as poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene imines), poly(caprolactones), poly(hydroxyalkanoates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof. In some embodiments the particle contains biodegradable polyesters or polyanhydrides such as poly(lactic acid), poly(glycolic acid), and poly(lactic-co-glycolic acid).

The particles can contain one or more amphiphilic polymers. Amphiphilic polymers can be polymers containing a hydrophobic polymer block and a hydrophilic polymer block.

The hydrophobic polymer block can contain one or more of the hydrophobic polymers above or a derivative or copolymer thereof. The hydrophilic polymer block can contain one or more of the hydrophilic polymers above or a derivative or copolymer thereof. In some embodiments the amphiphilic polymer is a di-block polymer containing a hydrophobic end formed from a hydrophobic polymer and a hydrophilic end formed of a hydrophilic polymer. In some embodiments, a moiety can be attached to the hydrophobic end, to the hydrophilic end, or both. The particle can contain two or more amphiphilic polymers.

The particles may contain one or more lipids or amphiphilic compounds. For example, the particles can be liposomes, lipid micelles, solid lipid particles, or lipid-stabilized polymeric particles. The lipid particle can be made from one or a mixture of different lipids. Lipid particles are formed from one or more lipids, which can be neutral, anionic, or cationic at physiologic pH. The lipid particle, in some embodiments, incorporates one or more biocompatible lipids. The lipid particles may be formed using a combination of more than one lipid. For example, a charged lipid may be combined with a lipid that is non-ionic or uncharged at physiological pH.

The particle can be a lipid micelle. Lipid micelles for drug delivery are known in the art. Lipid micelles can be formed, for instance, as a water-in-oil emulsion with a lipid surfactant. An emulsion is a blend of two immiscible phases wherein a surfactant is added to stabilize the dispersed droplets. In some embodiments the lipid micelle is a microemulsion. A microemulsion is a thermodynamically stable system composed of at least water, oil and a lipid surfactant producing a transparent and thermodynamically stable system whose droplet size is less than 1 micron, from about 10 nm to about 500 nm, or from about 10 nm to about 250 nm. Lipid micelles are generally useful for encapsulating hydrophobic active agents, including hydrophobic therapeutic agents, hydrophobic prophylactic agents, or hydrophobic diagnostic agents.

The particle can be a liposome. Liposomes are small vesicles composed of an aqueous medium surrounded by lipids arranged in spherical bilayers. Liposomes can be classified as small unilamellar vesicles, large unilamellar vesicles, or multi-lamellar vesicles. Multi-lamellar liposomes contain multiple concentric lipid bilayers. Liposomes can be used to encapsulate agents, by trapping hydrophilic agents in the aqueous interior or between bilayers, or by trapping hydrophobic agents within the bilayer.

The lipid micelles and liposomes typically have an aqueous center. The aqueous center can contain water or a mixture of water and alcohol. Suitable alcohols include, but are not limited to, methanol, ethanol, propanol, (such as isopropanol), butanol (such as n-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

The particle can be a solid lipid particle. Solid lipid particles present an alternative to the colloidal micelles and liposomes. Solid lipid particles are typically submicron in size, i.e. from about 10 nm to about 1 micron, from 10 nm to about 500 nm, or from 10 nm to about 250 nm. Solid lipid particles are formed of lipids that are solids at room temperature. They are derived from oil-in-water emulsions, by replacing the liquid oil by a solid lipid.

Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DUPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

Suitable cationic lipids include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N—(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

Suitable solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, for example, 12-30 carbon atoms, such as cetostearyl alcohol. Solid lipids can include higher fatty acids of 10-40, for example, 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40, for example, 12-30 carbon atoms, such as glyceryl monostearate, glycerol behenate, glycerol palmitostearate, glycerol trilaurate, tricaprin, trilaurin, trimyristin, tripalmitin, tristearin, and hydrogenated castor oil. Suitable solid lipids can include cetyl palmitate, beeswax, or cyclodextrin.

Amphiphilic compounds include, but are not limited to, phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), for example, between 0.1-30 (weight lipid/w polymer). Phospholipids that may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

### Pharmaceutical Compositions

Also provided are compositions comprising red blood cell ghosts (i.e. resealed RBCMFs) prepared by the methods described herein as well as compositions comprising a population of particles functionalized with RBCMFs prepared by the methods described herein. These compositions can be used, for example, for drug delivery or diagnostics or combination of the two (e.g., depending on the active agent(s) present in the resealed RBCMFs and/or the particles functionalized with RBCMFs).

The compositions described herein can include red blood cell ghosts and/or particles functionalized with RBCMFs in combination with pharmaceutically acceptable carriers. As would be appreciated by one of skill in this art, the carriers may be chosen based on the route of administration as described below, the location of the target issue, the drug being delivered, the time course of delivery of the drug, etc.

The pharmaceutical compositions of this disclosure can be administered to a patient by any means known in the art including oral and parenteral routes. The term “patient,” as used herein, refers to humans as well as non-humans, including, for example, mammals, birds, reptiles, amphibians, and fish. For instance, the non-humans may be mammals (e.g., a rodent, a mouse, a rat, a rabbit, a monkey, a dog, a cat, a primate, or a pig). In certain embodiments parenteral routes are desirable since they avoid contact with the digestive enzymes that are found in the alimentary canal. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays).

In a particular embodiment, the red blood cell ghosts and/or particles functionalized with RBCMFs of the present disclosure are administered to a subject in need thereof systemically, e.g., parenterally, or by IV infusion or injection.

The present invention may be understood more readily by reference to the following detailed description of various aspects of the invention and the examples included therein and to the Figures and their previous and following description.

### EXAMPLES

The invention will be described in greater detail by way of specific examples. The following examples are offered for illustrative purposes and are not intended to limit the invention in any manner. Those of skill in the art will readily recognize a variety of non-critical parameters which can be changed or modified to yield essentially the same results.

### Example 1: Scalable Manufacturing Platform for the Purification of Size-Controllable Human Red Blood Cell Membrane Fragments Via Tangential Flow Filtration and their Subsequent Ability to Form Resealed Red Blood Cell Ghosts and to Surface Coat Particles

Therapeutic nanoparticles (NPs) are subject to clearance by the mononuclear phagocytic system, which limits their in vivo performance. To counteract immune system recognition of NPs and non-specific protein adsorption on the NP surface, red blood cell membrane fragments (RBCMFs) were recently studied as a biocompatible NP surface coating to address these shortcomings. Biomimetic RBCMF surface coated therapeutic NPs may greatly enhance therapeutic efficacy by increasing the circulatory half-life of the therapeutic. RBCMFs are primarily purified via red blood cell (RBC) lysis followed by repeated centrifugation, pelleting, and resuspension of the lysed RBCs in a wash solution in order to produce RBCMFs devoid of cell-free hemoglobin (Hb). However, this purification process is not scalable due to the limits of scale of centrifugation. Therefore, there is a need for scalable processes to enable large scale production of RBCMFs for potential biomedical applications. Additionally, the RBCMF size should be tunable in order to be able to coat various types of therapeutic NPs for biomedical applications ranging from diagnostics, imaging, and drug delivery. In this study, we developed a highly scalable and reproducible process for purifying RBCMFs. Tangential flow filtration (TFF) was used to remove >99% of the cell-free Hb from lysed RBCs, along with other intracellular contents, resulting in RBCMFs with relatively high purity. RBCMFs possessed an initial size distribution ranging from ~2.3-3.0 μm, with some smaller species in the range of 600-800 nm. In order to further reduce the size of RBCMFs and homogenize the material, we developed a sonication protocol that reduced the sizes of RBCMFs down to ~50-70 nm. Both sonicated and unsonicated RBCMFs exhibited zeta (ζ) potentials in the range of −30 to −40 mV, making them well-suited for NP surface coating applications due to their high negative surface charge. No significant changes were observed in the ζ potential of −80° C. frozen RBCMFs thawed at 4° C., 20° C., and 37° C. after one freeze-thaw cycle, suggesting that RBCMFs may be stored at −80° C. without compromising their surface electrostatic properties. Finally, we evaluated the ability of sonicated and unsonicated RBCMFs to both coat particles and to form resealed RBC ghosts. Sonicated RBCMFs were able to coat positively charged glass beads, while resealed RBCMFs containing Hb exhibited similar viscosity, oxygen equilibria, oxygen offloading rate, and volume fraction as native RBCs when formulated at an equivalent hematocrit. Taken together, this work describes a novel scalable process for manufacturing RBCMFs for potential biomedical applications.

### Introduction

A critical challenge in the development of therapeutic nanoparticles (NPs) is their recognition by the reticuloendothelial system, and subsequent clearance from the circulation. The foreign surface properties of NPs lead to macrophage uptake and rapid clearance, which significantly reduces their circulatory half-life and therapeutic efficacy. These limitations are prevalent in many biomedical NP applications including drug delivery, diagnostics, and imaging. Therefore, various synthetic materials have been used to shield the NP surface to evade immune recognition. NP surface conjugation with poly (ethylene glycol) (PEG) is known to increase NP residence time, due to its hydrophilic surface properties. Unfortunately, there are concerns regarding the immunocompatibility of PEG. Some patients have preexisting PEG antibodies or develop an immune response after repeated exposure to PEG. Polydopamine (PDA) surface coatings have also been studied and seem to offer anti-inflammatory properties by scavenging reactive oxygen species (ROS). However, there is a risk of forming non-covalently bound oligomers of dopamine, which do not attach to the NP surface and fail to confer benefits.

Recently, the use of red blood cell (RBC) membrane fragments (RBCMFs) to coat the surface of NPs has emerged as a robust and versatile approach for integrating natural and synthetic biomaterials to form functional NPs that evade immune recognition, since these coated NPs mimic the surface of RBCs which are not recognized by the body's immune system. Purification of RBCMFs can involve RBC lysis and the removal of cell-free hemoglobin (Hb) from the lysed RBCs, since cell-free Hb is cytotoxic. Centrifugation is primarily used to remove cell-free Hb from lysed RBCs, which unfortunately is not scalable. Once the RBCMFs have been purified, the size of the resulting RBCMFs needs to be reduced to standardize the RBCMF size to facilitate uniform coating of NP surfaces.

In this Example, we developed an easy and scalable method for preparing Hb-free RBCMFs. Tangential flow filtration (TFF) was used to remove cell-free Hb from lysed RBCs in the permeate stream, while retaining RBCMFs in the retentate. Additionally, we implemented a subsequent sonication step to further reduce the size of RBCMFs to improve its consistency for it to function as a suitable NP coating. We then evaluated the ability of sonicated and unsonicated RBCMFs to coat particles and encapsulate protein, respectively. We demonstrated that fluorescently tagged sonicated RBCMFs can be used to coat small microbeads, while unsonicated RBCMFs were resealed with Hb and their biophysical properties compared to native RBCs.

### Materials and Methods

Materials. Non-irradiated expired packed human RBC (PRBC) units were obtained from The Ohio State University Wexner Center Office of Transfusion Services (Columbus, OH). Size 17 silicone and BPT tubing were purchased from Cole-Parmer (Vernon Hills, IL). Hollow fiber (HF) TFF modules with 0.65 μm molecular weight cut-off (MWCO) were obtained from Repligen (Rancho Dominguez, CA). Hydrochloric acid (HCl), sodium hydroxide (NaOH), sodium chloride (NaCl), sodium phosphate monobasic (NaH₂PO₄), α-cyano-4-hydroxy-cinnamic acid MALDI matrices, and sodium phosphate dibasic (Na₂HPO₄) were purchased from Sigma-Aldrich (St. Louis, MO). A CL-334 Sonicator and Q500 probe were sourced from Qsonica (Newton, CT). HPLC-grade chloroform and methanol were purchased from Fisher Scientific (Waltham, MA).

RBC Wash. PRBCs were washed in accordance with the method described by Lu et al. PRBCs were diluted to 45% hematocrit and washed with 0.9% wt NaCl via a 0.65 μm TFF module and a centrifugal pump to remove cell-free Hb and other proteins from the RBCs. RBCs were then subjected to 10 constant volume diacycles with 0.9% wt NaCl as the wash solution.

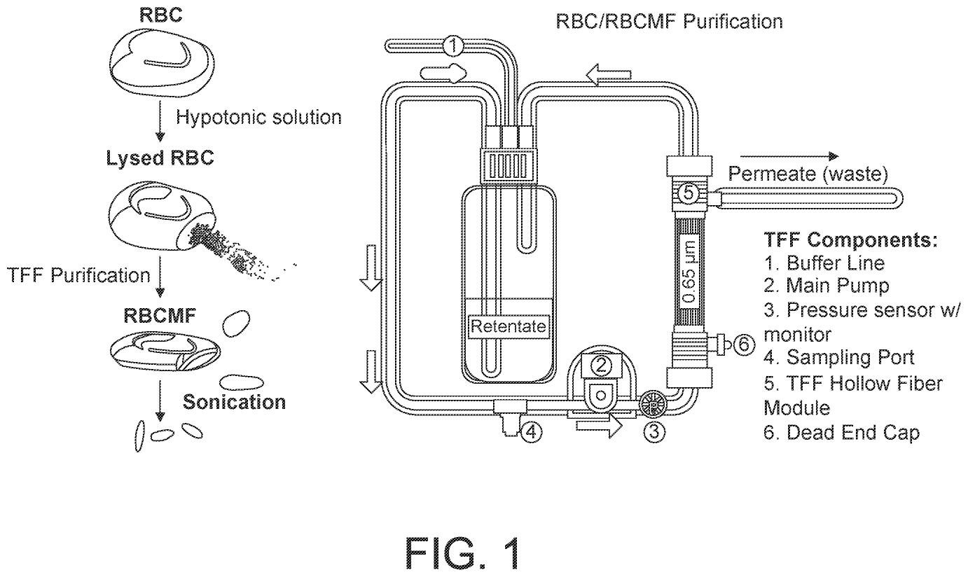

RBC Lysis and RBCMF Purification. Washed RBCs were lysed using an equal volume of deionized (DI) water to yield RBCMFs and cell-free Hb. The total volume of the RBCMF solution was then reduced to −400 mL, and then subjected to 10 diacyles of constant volume diafiltration on a 0.65 μm TFF module to remove intracellular debris and cell-free Hb using phosphate buffer (5 mM, pH 8.0) as the wash solution. FIG. 1 shows the filtration setup for both the RBC washing and RBCMF filtration processes. After each diacycle, permeate and retentate samples were collected for analysis. The cell-free Hb concentration of permeate samples were analyzed using the method developed by Winterbourn, while retentate samples were subject to two analysis steps. First, the cell-free Hb concentration in the retentate was measured using the Winterbourn assay. Second, particle size analysis was performed using a Coulter Counter (Beckman Coulter, Brea, Ca). After 10 diacycles, the final RBCMFs were concentrated down to a volume of −100 mL and stored at −80° C. for further processing and analysis. The final concentration of RBCMFs was measured via Coulter Counter (CC) analysis and was −3.5×10⁹ RBCMFs/mL. Statistical analysis was then used to determine the number of diacycles necessary before the amount of cell-free Hb removed from the retentate and permeate ceased to be significant compared to the previous diaycle. This would determine the stage at which, statistically, no more Hb would be removed from the system.

Sonication of RBCMFs. To further reduce the size and increase the consistency of RBCMFs for possible NP coating applications, sonication was employed using a Qsonica CL-334 ultrasonicator. Briefly, 50 mL of unsonicated RBCMFs were placed in a small beaker, which was then placed in an ice bath. RBCMFs were sonicated using a Q500 half-inch probe tip, with the vessel placed in an ice bath to prevent protein denaturation. The sonication profile consisted of an amplitude of 70%, with pulses of 2 s on and 1 s off, repeated over three minutes. This process was repeated depending on the final desired size of the RBCMFs; six total cycles yielded an average RBCMF size of −50 nm, while a greater number cycles was found to further reduce RBCMF size.

Freeze-Thaw Study of RBCMFs. To determine the freeze-thaw stability of RBCMFs, 2 mL samples of sonicated and unsonicated RBCMFs were collected and frozen at −80° C. Samples were then thawed at 4° C., 20° C., and 37° C. Particle size distribution and ζ potential analysis were performed on all fresh and thawed RBCMFs.

ζ Potential Analysis. ζ potential analysis was performed on all fresh and thawed RBCMFs using a Brookhaven Instruments ZetaPals instrument (Holtsville, NY) for phase-shift-based analysis light scattering. Analysis was done both for sonicated and unsonicated samples. RBCMFs were diluted 100× to a concentration of ~2×10⁷ RBCMFs/mL in DI water.

Dynamic Light Scattering (DLS). The diameter of RBCMF particles <1 μm in diameter was measured using a BI-200SM goniometer (Brookhaven Instruments Corp., Holtsville, NY) at an angle of 90° and wavelength of 637 nm. RBCMFs were diluted 100×, and unsonicated samples were filtered through a 1.2 μm poly(ether) sulfone (PES) syringe filter. This last filtration step ensured a better signal to noise ratio for the smaller RBCMF species present in unsonicated RBCMFs, with CC analysis used to measure larger diameter RBCMF species (>1 μm). Measuring unsonicated RBCMFs without filtration resulted in a decrease in the accuracy of measuring smaller particle diameters, since DLS is less accurate when measuring particles much larger than 1 μm.

Transmission Electron Microscopy (TEM) Analysis. The morphology of RBCs and RBCMFs was analyzed using a FEI Tecnai G2 Biotwin transmission electron microscope (TEM) (FEI, Hillsboro, OR), and the particle diameter was measured from the TEM images using Image J (NIH). All samples were diluted 100× in DI water.

Coulter Counter Size Analysis. To analyze the size distribution of RBCMFs on the micron scale, Coulter Counter (CC) analysis was performed on a 100× dilution of the sample, which was further diluted by adding 0.2 mL of the diluted sample into 20 mL of double-filtered Isoton-III buffer (Beckman Coulter, Brea, CA). Particle counts and size distribution data were collected from each sample, and each sample was prepared identically.

Quantification of Hb Concentration. The cell-free Hb concentration of RBC, permeate, and retentate samples was measured using the Winterbourn assay. Samples were diluted to <1 mg/mL Hb, and 1 mL was pipetted into a disposable cuvette. The absorbance spectra of RBC/RBCMF supernatants was measured using an OLIS 8452A diode array spectrophotometer (OLIS, Athens, GA). Due to the fact that large particles scatter light, retentate samples were additionally centrifuged and sterile-filtered through a 0.2 μm Poly-Ether Sulfone filter. This was acceptable, since the assay aims to quantify the amount of cell-free Hb present in the solution.

Unsonicated RBCMF Resealing Process to Form RBC Ghosts. Resealing of unsonicated RBCMFs to form RBC ghosts (RBCGs) was achieved by mixing a hypertonic saline solution with RBCMFs suspended in hypotonic solution. Aliquots of 4M NaCl and 4M KCl, taken from stock solutions, were added via syringe pump to stirred RBCMFs to achieve 5× physiological salt levels, thus creating a hypertonic environment. The mixture was cooled for 5 minutes at −20° C., and then incubated at 37° C. for 60 minutes. The resulting RBCGs were centrifuged and had their volume fraction adjusted to ~45% before being stored at 4° C. for further analysis.

To assess the feasibility of using the resealed RBCGs as a potential RBC substitute or drug delivery vehicle, Hb was encapsulated inside the RBCGs by mixing the RBCMFs with physiological concentrations of Hb before the addition of the hypertonic salt solution to form Hb encapsulated RBCGs. Upon forming RBCGs encapsulating Hb, centrifugation was used to remove cell-free Hb from the RBCGs encapsulated Hb with isotonic PBS as the wash solution. After cell-free Hb was removed, the Hb encapsulation efficiency of RBCGs was measured by determining the amount of Hb lost. The percentage of encapsulated Hb retained was normalized to the final volume fraction of RBCGs, to account for any batch-to-batch inconsistencies in the RBCMF purification process. After measuring total Hb loss, the final volume fraction of RBCGs was adjusted to ~45% and its' biochemical and biophysical properties compared to an RBC solution at the same volume fraction. As a control, ‘blank’ RBCGs that contained no Hb were resealed and used to directly compare to Hb-containing RBCGs. A total of seven replicates of Hb-containing RBCGs and empty RBCGs were resealed and further analyzed.

Oxygen Equilibrium and Oxygen Offloading Analysis. To assess the ability of Hb encapsulated RBCGs to bind oxygen, the oxygen equilibrium curve (OEC) and the kinetics of oxygen offloading were measured using a Hemox Analyzer (TCS Scientific, New Hope, PA) and stop-flow UV-visible spectrometer (Applied Photophysics Ltd., Surrey, UK), respectively. The OEC was measured and analyzed via the Hill equation to regress the partial pressure of oxygen (pO₂) where 50% of the Hb is saturated with oxygen (P₅₀) and the Hill coefficient (n). RBCGs containing Hb were compared against empty RBCGs, and both groups were compared to the OECs for cell-free Hb and fresh RBCs obtained from the literature. Oxygen offloading kinetics were measured and analyzed to regress the oxygen offloading rate constant (k_{off,O2}). Similar to the OEC measurements, cell-free Hb, RBCs, and empty RBCGs were used as controls to compare against Hb-containing RBCGs.

Coulter Counter Size Distribution and Concentration Analysis of RBCGs. To analyze the size distribution and concentration of RBCGs, CC analysis was performed on a 100× dilution of the sample, which was further diluted by adding 0.2 mL of the diluted sample into 20 mL of double-filtered Isoton-III buffer (Beckman Coulter, Brea, CA). Particle counts and size distribution data were collected from each sample, and each sample was prepared identically. The protocol used was similar to one established for RBCs and was adapted to for RBCGs.

Viscosity Analysis. The viscosity of Hb-containing RBCGs and blank RBCGs were compared to unmodified RBCs using a Brookfield-Ametek LV Rheometer (Middleborough, MA). 500 μL of sample was loaded into the rheometer, and the viscosity at a shear rate of 160 s⁻¹ was measured at 37° C.

Sonicated RBCMF Coating of Microparticles. To test the feasibility of sonicated RBCMFs as a particle coating, a protocol similar to the one established by Walters et al. was used to coat 30 μm diameter glass microbeads with fluorescently-tagged RBCMFs. The beads were first functionalized to possess a positive surface charge by using Zetag (Solenis, Wilmington, DE), and then incubated for 12 hours with sonicated RBCMFs that had been stained with fluorescently labelled wheat germ agglutinin (WGA), which binds to the glycoproteins present on the RBCMF cell membrane. At the end of the incubation period, the particles were set in formaldehyde and excess formaldehyde/RBCMFs were removed. The WGA had red fluorescence, and both a green-fluorescent Zetag as well as a non-fluorescent Zetag were tested to achieve better image contrast.

Statistical Analysis. The statistical significance of the ζ potential of RBCMFs at different thaw temperatures was evaluated using one-way ANOVA. Significant differences in cell-free Hb removal using TFF was determined using one-way ANOVA testing between diacyles, and a follow-up Tukey-Kramer test was performed post-hoc to determine which diacycle groups were significantly different from each other. A one-way ANOVA test was also used to determine differences in biophysical properties between empty RBCGs, Hb-loaded RBCGs, washed RBCs, and cell-free Hb. Statistical significance was achieved if p<0.05. The SPSS software package was used for all statistical analysis (IBM, Armonk, NY).

### Results and Discussion

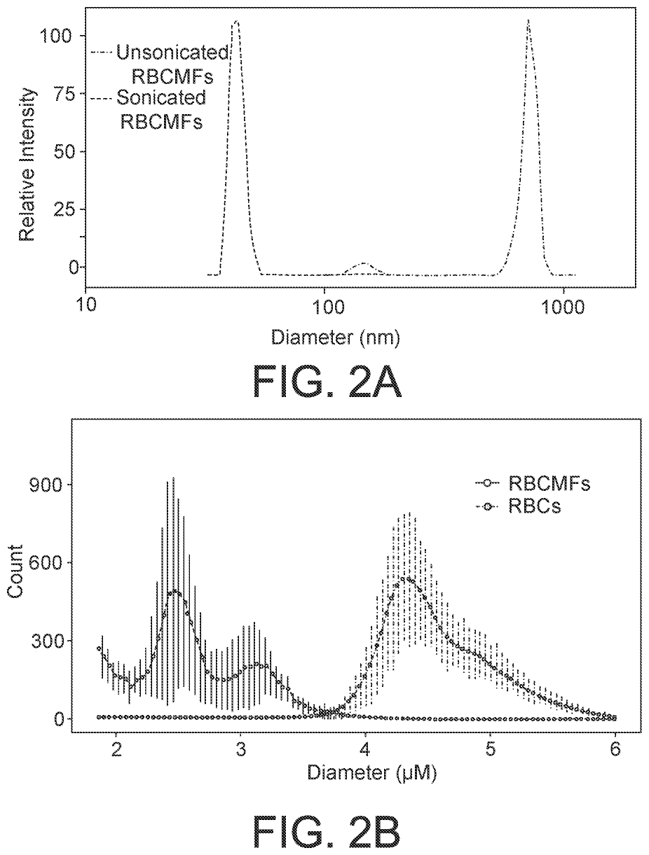

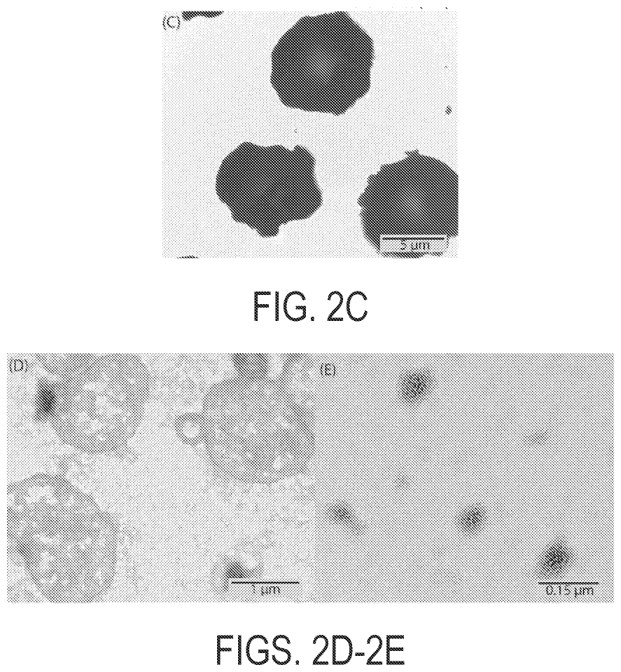

RBCMF Size Measurements 3.1.1 RBCMF Hydrodynamic Diameter and Morphology. DLS analysis showed a clear difference between the hydrodynamic diameter of sonicated and unsonicated RBCMFs. After filtering unsonicated RBCMFs through a 1.2 μm filter to remove large diameter RBCMFs, the lower size distribution ranges from 600-800 nm. Table 1 summarizes the observed size ranges for RBCMFs using both CC and DLS. The RBCMF purification scheme exposes RBCMFs to shear stress, which most likely contributed to the smaller size distribution of RBCMFs compared to RBCs. After sonicating RBCMFs for 12 minutes, the resulting membrane fragments were monodisperse, with mean sizes in the range of 40-70 nm. The change in size distribution is shown in FIG. 2A, which contains representative size distributions for both sonicated and unsonicated RBCMFs. The mean particle diameter measured via DLS for all replicates was 58.6±3.8 nm for sonicated particles and 677±129 nm for unsonicated RBCMFs, which were statistically different (p<0.05). The majority of unsonicated RBCMFs were in the range of 1.7-3.2 μm.

TABLE 1
Particle size of RBCMFs.

| Sample Type | Average DLS size (nm) | Mean CC size (μm) |
|---|---|---|
| Unsonicated RBCMFs | 677 ± 129* | 2.699 ± 0.0508 |
| Sonicated RBCMFs | 58.6 ± 3.8 | N/A** |

(*Filtered using a 1.2 μm glass-fiber filter to increase the signal to noise ratio.
**Particles too small to be measured via CC analysis.)

TEM was used to probe the morphology of RBCs and RBCMFs at various stages in the RBCMF manufacturing process. FIG. 2C shows washed RBCs, and subsequent FIGS. 2D and 2E demonstrate the morphological differences that RBCMFs and sonicated RBCMFs exhibit, respectively. FIG. 2D demonstrates the removal of cell-free Hb from RBCMFs following the TFF purification step, which reduces TEM image contrast due to the removal of Hb from the RBCMFs. It also demonstrates the reduction in size of RBCMFs from intact RBCs that are shown in FIG. 2B. Finally, FIG. 2E demonstrates the effectiveness of the sonication process, which reduced the size of the RBCMFs in the range 40-70 nm. This reduction in size complements the associated reduction in size observed in FIG. 2A for sonicated RBCMFs via DLS.

RBCMF Coulter Counter Size Analysis. After RBC hemolysis and TFF purification of RBCMFs, a significant reduction in particle size was observed for RBCMFs compared to intact RBCs. FIG. 2B summarizes these changes, which were obtained with N=9 statistical replicates. RBCs possessed diameters ranging from 4-5.3 μm, which is smaller than reported values for RBCs, but consistent with measurements of RBCs using CC. RBCMFs exhibited a size distribution primarily between 2-3.5 μm. Measuring sonicated RBCMFs using CC yielded results indistinguishable from blank Isoton-III runs, which signify that the RBCMFs are well below the 600 nm cutoff measurement range of the CC.

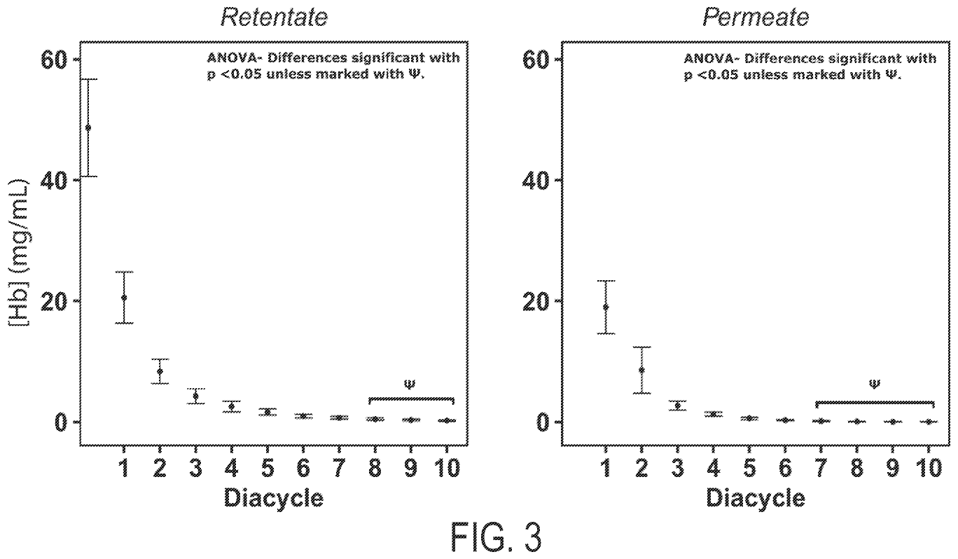

Cell-Free Hb Removal from RBCMFs. After RBC lysis, the concentration of cell-free Hb in the permeate decreased from an average of 19.0±2.2 mg/mL to 0.04±0.01 mg/mL after 10 diacycles. Similarly, the cell-free Hb concentration in the retentate decreased from 48.7±4.0 mg/mL to 0.3±0.07 mg/mL. FIG. 3 shows the reduction in cell-free [Hb] with increasing diacycles. Both reductions (i.e., permeate and retentate) in cell-free Hb levels were statistically significant (p<0.05) when comparing initial and final cell-free Hb concentrations to each other. The RBCMF purification protocol demonstrates the ability to remove >99% of the cell-free Hb present in the initially lysed RBC solution. The 0.65 μm HF membrane was effective in removing cell-free Hb and other debris below its MWCO, while retaining RBCMFs larger than the MWCO. Similar findings were observed for all replicates (N=10). Differences in cell-free Hb retentate concentrations were significant (p<0.05) until after the eighth diacycle, where subsequent diacycles fail to yield significant changes in the cell-free Hb concentration. The permeate cell-free Hb differences were significantly different until after the seventh diacycle, where again subsequent diacycles failed to yield significant reduction in the cell-free Hb concentration.

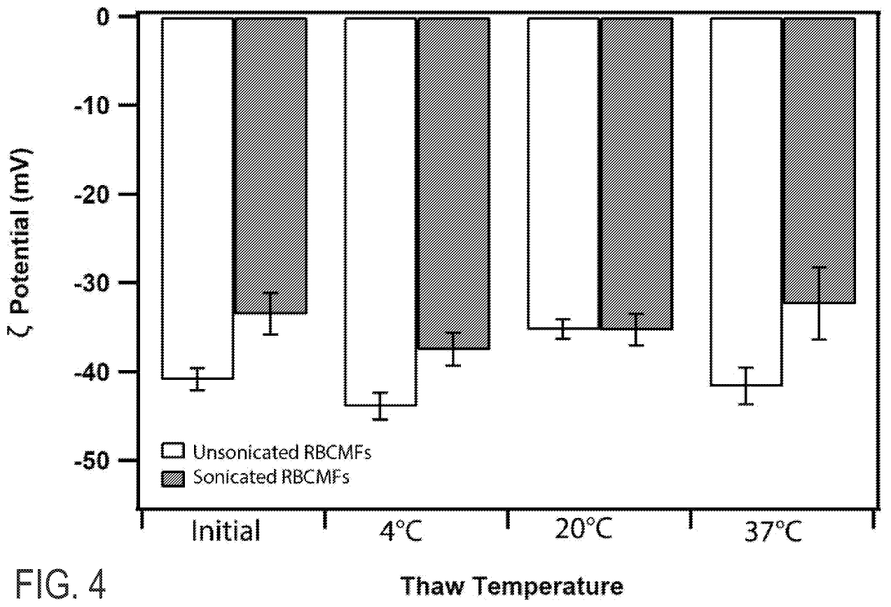

Zeta Potential of RBCMFs. The ζ potential of fresh sonicated and unsonicated RBCMFs were found to be −33.5±2.3 mV and −40.8±1.2 mV, respectively. These two values were not significantly different from each other (p>0.05). Thawing of frozen RBCMFs at 4° C. lowered the ζ potential of sonicated and unsonicated RBCMFs to −37.4±1.8 mV and −43.8±1.5 mV respectively. Thawing of frozen RBCMFs at 20° C. raised the ζ potential of the sonicated and unsonicated RBCMFs to −35.3±1.8 mV and −35.2±1.1 mV, while thawing of frozen RBCMFs at 37° C. results in ζ potentials of −32.3±4.0 mV and −41.6±2.0 mV for sonicated and unsonicated RBCMFs, respectively. FIG. 4 summarizes these changes. ANOVA analysis resulted in p-values >0.05. As a result, we could not conclude that a single freeze-thaw cycle meaningfully alters the ζ potential from the initially formed RBCMFs, which were not subject to a freeze-thaw cycle. Therefore, subjecting RBCMFs to a single freeze-thaw cycle did not significantly alter the ζ potential when compared to control values. This is useful in assessing the feasibility of using RBCMFs as NP coatings after extended time in storage.

These ζ potentials are more negative than reported ranges of −9.3 to −15 mV for fresh RBCs, which are consistent with a previous study that observed that the ζ potential is lower for RBCMFs than intact RBCs. After hemolysis, membrane proteins on the inside leaflet of the RBC membrane are exposed to the extracellular space. Some of these exposed proteins have associated negative charges, which may explain why there is a measured change from physiological RBC ζ potential values.

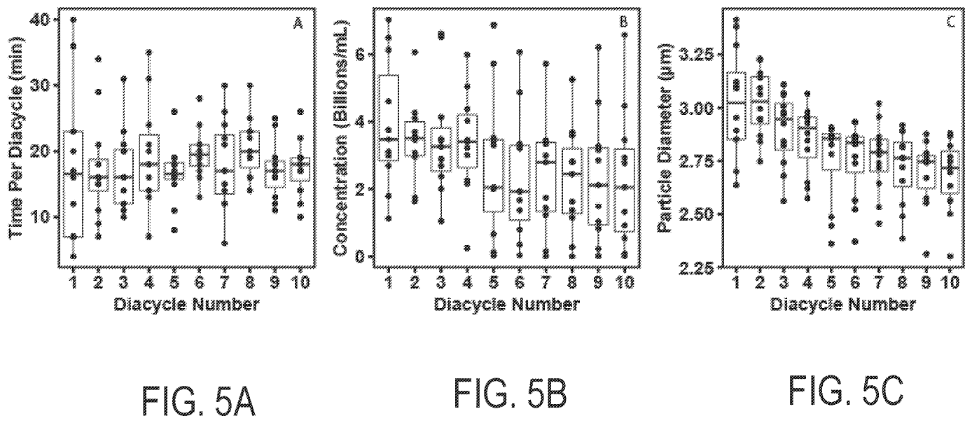

Effect of TFF on diacycle time and RBCMF concentration and size. Analysis of the individual TFF diacycles required to remove >99% Hb yielded insights into the effects of the TFF washing process on the diacycle time and RBCMF concentration and size, and these findings are summarized in FIGS. 5A-5C, with n=11 replicates. As shown in FIG. 5A, the time required to complete each diacycle did not significantly increase with increasing number of diacycles (p>0.05). Outside factors such as the number of times each TFF cartridge had been previously used had an impact on batch-to-batch diacycle times, but overall no significant change in diacycle time was observed, both within each replicate and when combining all replicates together. Additionally, no significant change in RBCMF concentration was observed with increasing number of diacycles (p>0.05), and particle size was only slightly impacted. Specifically, diacycles 1 and 2 had significantly larger RBCMFs than diacycles 6-10 (p<0.05), though no other significant size differences across any of the groups were detected. FIGS. 5B and 5C highlight these trends and seem to suggest that the shear forces applied in TFF circuit do not significantly alter the concentration and size of RBCMFs for the 10 diacycles required to remove 99% of the cell-free Hb. The lack of change in RBCMF concentration and only slight decrease in particle size suggest that TFF can acceptably perform its major function of removing cell-free Hb without significantly altering the properties of the resulting RBCMFs.

Resealing of RBCGs using unsonicated RBCMFs; Hb retention in RBCGs. Resealed RBCGs demonstrated the ability to encapsulate Hb after three centrifugation cycles in which unencapsulated cell-free Hb was removed from RBCG-encapsulated Hb. On average, the Hb retention rate was 14.78%±5.92% compared to the initial amount of Hb to be encapsulated. However, this does not account for batch-to-batch variations associated with purification of RBCMFs. As a result, a more accurate normalized Hb retention rate may be employed by dividing the absolute Hb retention rate of the RBCGs by their resultant volume fraction. This results in a normalized Hb retention factor of 0.86±0.24%/%. The normalized Hb retention factor indicates the effectiveness of RBCGs at retaining encapsulated material as a function of the volume fraction of RBCGs in the final product. For example, for every 1% volume fraction of RBCGs, there will be on average 0.86% of the encapsulated material retained. Therefore, a normalized Hb retention rate of 1%/% would suggest that the RBCGs are perfectly encapsulating the target material (i.e., Hb). It should be noted that this normalized Hb retention factor takes into account RBCGs that have become unsealed due to the centrifugation process and have subsequently lost their Hb, and this may be an explanation for why the observed normalized Hb retention factor is only 0.86%/%. Future studies will focus on optimizing this retention factor by utilizing a less-damaging method for cell-free Hb removal that minimizes potential damage to RBCGs.

RBCG Viscosity, Size, and Concentration. As summarized in Table 2, both Hb-loaded RBCGs and empty RBCGs did not have significant differences in viscosity or concentration compared to native RBCs, when formulated at the same hematocrit of 45%. However, both types of RBCGs were significantly smaller (p<0.05) than native RBCs and significantly larger (p<0.05) than cell-free Hb. The two RBCG groups (Hb-loaded RBCGs and empty RBCGs) did not have significantly different mean sizes or concentrations from each other. This was expected, as both RBCG samples were prepared very similarly, with the main difference being the presence or absence of encapsulated Hb. Overall, RBCGs maintained similar viscosity levels to native RBCs when adjusted to a 45% volume fraction, which matches the average male human hematocrit. This lack of significant difference in viscosity is important, as systemically introducing fluids that have much higher viscosity than blood can lead to increased blood pressure.

TABLE 2
Viscosity, size and concentration data for RBCGs, Hb,
and RBCs. RBCGs-Hb and RBCGs-Blank were both formulated
at a hematocrit (i.e. volume fraction) of 45%.

| | Viscosity | Size | Concentration |
|---|---|---|---|
| | (cP) | (μm) | (1E9/mL) |
| RBCGs-Hb | 7.079 ± 1.041 | 1.844 ± 0.132 | 4.368 ± 0.492 |
| RBCGs-Blank | 5.169 ± 0.306 | 1.843 ± 0.095 | 6.825 ± 1.339 |
| Cell-Free Hb | Dependent on | 0.005 | Variable |
| | Concentration | | |
| RBCs | 3.93 ± 1.77 | 4.65 ± 0.6 | 4.625 ± 0.35 |

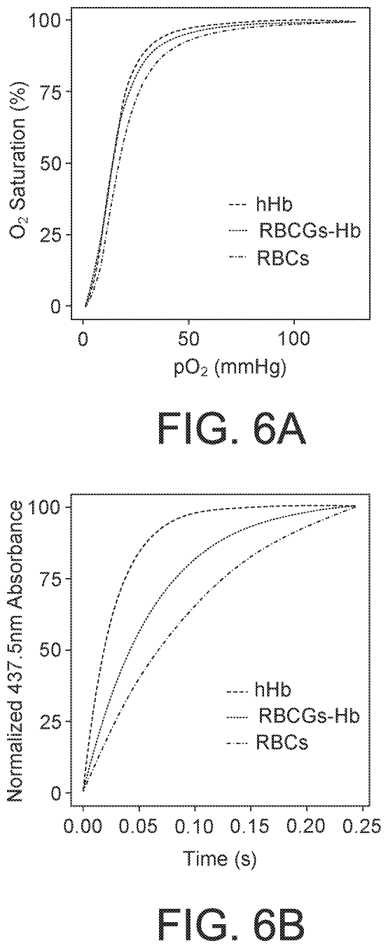

Oxygen Equilibrium and Offloading. Hb-containing RBCGs demonstrated the ability to bind and release oxygen as shown in Table 3 and FIGS. 6A-6B. The oxygen affinity (i.e. P₅₀, partial pressure of oxygen where half of the Hb is saturated with oxygen) and cooperativity of RBCGs-Hb were not significantly different from cell-free Hb (p>0.05), but was significantly lower in magnitude than native RBCs (p<0.05). This difference stems from the fact that the encapsulated Hb inside RBCGs does not contain the allosteric effector 2,3-bisphosphoglycerate, which is responsible for the high P₅₀ of fresh RBCs. This indicates that the resealed RBC membrane and additional encapsulated salts do not affect the performance of the encapsulated Hb in any meaningful way. RBCGs loaded with Hb significantly reduced the kinetics of oxygen offloading compared to cell-free Hb (p <0.05), due to the RBC membrane which acts as a diffusion barrier to oxygen transport. The data for RBCG-Hb oxygen equilibrium analysis and oxygen offloading kinetics were compared against cell-free Hb and RBCs found in the literature.

TABLE 3
Oxygen affinity (P₅₀), Hill coefficient (n), and oxygen offloading
rate constant (k_{off, O2}) for RBCGs, Hb, and RBCs.

| | k_{off, O2} (s⁻¹) | P₅₀ (mm Hg) | n |
|---|---|---|---|
| RBCGs-Hb | 13.046 ± 1.394 | 13.903 ± 0.318 | 2.370 ± 0.037 |
| Empty RBCGs | NA* | NA* | NA* |
| Cell-free Hb | 36.51 ± 2.53 | 12.55 ± 0.97 | 2.64 ± 0.09 |
| RBCs | 8.61 ± 2.55 | 27.16 ± 4.45 | 2.21 ± 0.13 |

*Empty RBCGs do not contain Hb and cannot bind and release oxygen.

### Microparticle Coating Using Sonicated RBCMFs

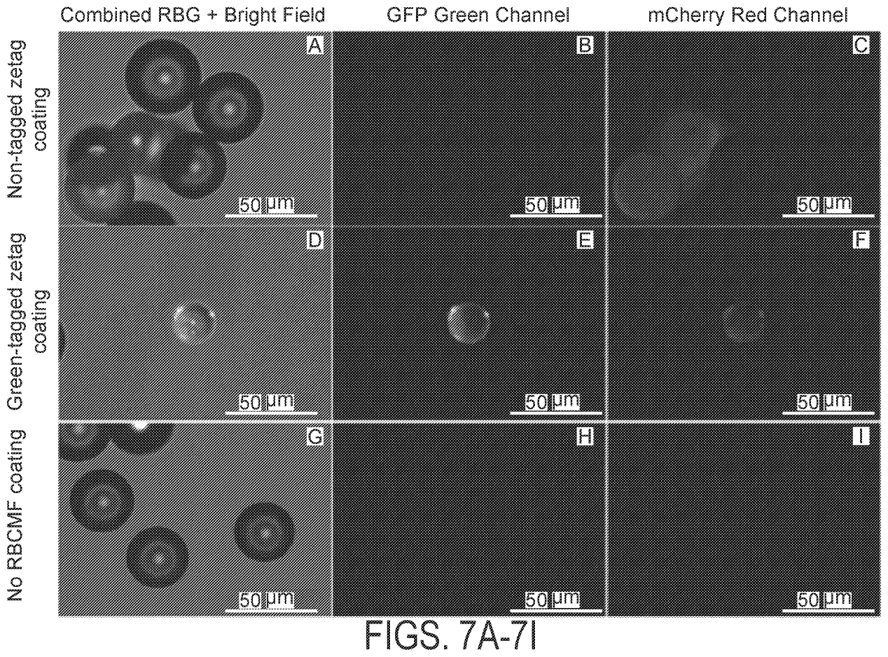

The sonicated RBCMFs demonstrated the ability to coat particles many orders of magnitude larger than them, as evidenced by the images shown in FIGS. 7A-7I. Sonicated RBCMFs bound to red fluorescently labelled WGA were able to be detected both with and without the green fluorescence of the zetag coated glass particles, removing a potential source of noise and confirming that it was only the fluorescently labeled sonicated RBCMFs that produced the red fluorescence observed in FIGS. 7A, 7C, 7D, and 7F. The presence of red-fluorescent WGA protein on the surface of the beads indicates good adhesion of the sonicated RBCMFs to the bead surface, as any unconjugated WGA would have been detected in the interstitial space between beads. FIGS. 7G-7I indicate that the beads themselves are not contributing to the fluorescence signal. Without the GFP associated with the zetag functional coating or the WGA-stained RBCMFs, there is no fluorescent signal, and the beads are only visible in bright-field imaging. In section 3.3, we have established that sonicated RBCMFs exhibited a negative ζ potential, so their attraction to the positively-charged zetag surface on the glass beads is expected. This demonstrates the potential of using sonicated RBCMFs for coating micro-/nano-particles.

### CONCLUSION

We demonstrate that TFF is a scalable process for the production of RBCMFs. The hydrodynamic diameter of RBCMFs was tunable via sonication. The purified RBCMFs exhibited favorable surface charge and size ranges for coating particles. Potential adverse effects due to the presence of cell-free Hb were mitigated by the removal of >99% of the cell-free Hb present in the RBCMF preparations via TFF. Finally, RBCMFs exhibited favorable long-term storage stability, since there was no significant change in ζ potential and hydrodynamic diameter before and after a freeze-thaw cycle.

In this work, we also demonstrated the ability of both sonicated and unsonicated RBCMFs to coat particles and form resealed RBCGs. The resealing of RBCGs via ionic reversal was shown to be a viable method for the self-assembly of RBCGs using the RBCMFs purified in this process. These sealed particles encapsulated Hb, while not significantly affecting many of the biochemical properties of the encapsulated Hb.

### Example 2. Tangential Flow Filtration Assisted Washing of Resealed Human Red Blood Cell Membrane Fragments Encapsulating Hemoglobin Improves Product Consistency and Hemoglobin Loading Compared to Centrifugation

### Overview

The biodistribution of many therapeutics is controlled by the immune system. In addition, some molecules are cytotoxic when not encapsulated inside of larger cellular structures, such as hemoglobin (Hb) encapsulation inside of red blood cells (RBCs). To counter immune system recognition and cytotoxicity, drug delivery systems based on red blood cell membrane fragments (RBCMFs) have been proposed as a strategy for creating immunoprivileged therapeutics. However, the use of RBCMFs for drug delivery applications requires purification of RBCMFs at high scale from lysed RBCs free of their intracellular components. In this study, we were able to successfully use tangential flow filtration (TFF) to remove >99% of cell-free Hb from lysed RBCs at high concentrations (30-40% v/v), producing RBCMFs that were 1.46±0.52 μm in diameter. We were also able to characterize the RBCMFs more thoroughly than prior work, including measurement of particle zeta potential, along with individual TFF diacycle data on the cell-free Hb concentration in solution and time per diacycle, as well as concentration and size of the RBCMFs. In addition to purifying RBCMFs from lysed RBCs, we utilized a hypertonic solution to reseal purified RBCMFs encapsulating a model protein (Hb) to yield resealed Hb-encapsulated RBC ghosts (Hb-RBCGs). TFF was then compared against centrifugation as an alternative method for removing unencapsulated Hb from Hb-RBCGs, and the effects that each washing method on the resulting Hb-RBCG biophysical properties was assessed. For both TFF and centrifugal washed Hb-RBCGs, the existence of RBCGs smaller than RBCs enhanced oxygen transport, resulting in oxygen equilibrium curves and oxygen release kinetics in between that of cell-free Hb and fresh RBCs. However, TFF washed Hb-RBCGs resulted in a ~27% increase in protein encapsulation efficiency compared to centrifugation, and the resultant TFF Hb-RBCGs had improved colloidal stability. Unfortunately, Hb-RBCGs washed via centrifugation elicited Hb-RBCG aggregation that could not be disrupted without partially lysing the Hb-RBCGs, further reducing the protein encapsulation efficiency. In summary, we demonstrate that TFF can be efficiently used to dually purify RBCMFs as well as RBCGs encapsulating protein in a simple scalable process.

### Materials and Methods

Materials. Hydrochloric acid (HCl), sodium hydroxide (NaOH), sodium chloride (NaCl), potassium chloride (KCl), sodium phosphate monobasic (NaH₂PO₄), and sodium phosphate dibasic (Na₂HPO₄) were purchased from Sigma-Aldrich (St. Louis, MO). Non-irradiated packed human RBC units were obtained from The Ohio State University Wexner Center Office of Transfusion Services (Columbus, OH) for purification of RBCMFs. The cells were expired, and thus unsuitable for transfusion. Size 16 and 17 silicone and biopharmaceutical tubing were purchased from Cole-Parmer (Vernon Hills, IL). Hollow fiber TFF modules with 0.65 μm and 500 kDa molecular weight cut-offs (MWCO) were obtained from Repligen (Rancho Dominguez, CA).

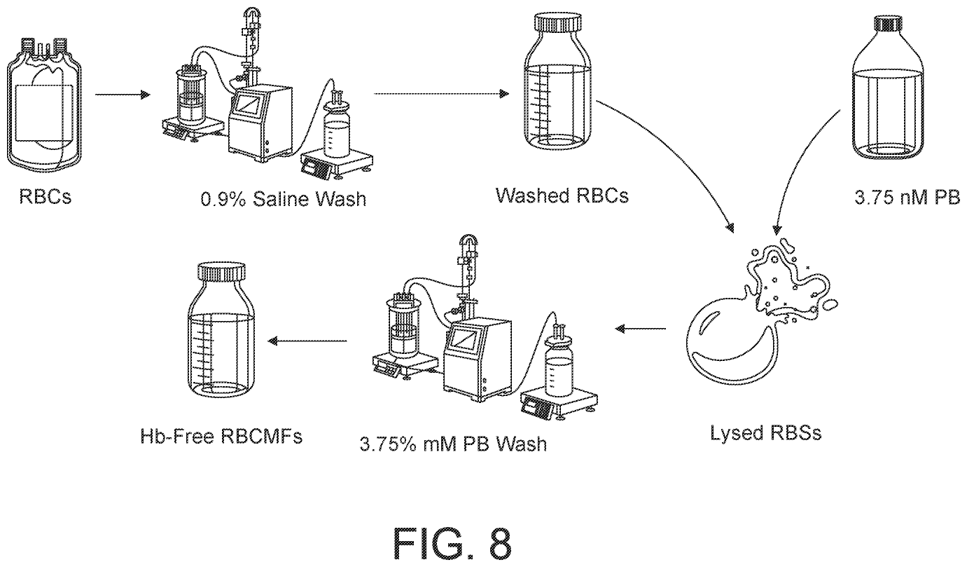

RBC Wash and Membrane Fragment Purification Process. RBCs were washed via TFF as described by Lu et al. RBCs were diluted to 45% volume fraction (hematocrit) and subjected to 10 constant volume buffer exchanges with 0.9 wt % NaCl as the wash solution using a 0.65 μm TFF module and a centrifugal pump to remove cell-free Hb and other cell-free proteins from the RBCs. The washed RBCs were lysed by mixing them with an equal volume of phosphate buffer (PB, 3.75 mM, pH 7.4), yielding a mixture RBCMFs and cell-free proteins. The total volume of the lysate was reduced to −300 mL and was subjected to 10 diacyles of constant volume diafiltration on a 0.65 μm TFF module to remove intracellular debris and cell-free Hb using PB as the wash solution. After complete removal of cell-free Hb, RBCMFs were concentrated to a minimum system volume of −80 mL. This final step was performed to maximize the RBCMF concentration for resealing, which would increase the Hb encapsulation efficiency of the resealed RBCGs. The RBC wash and RBCMF purification process is shown in FIG. 8.

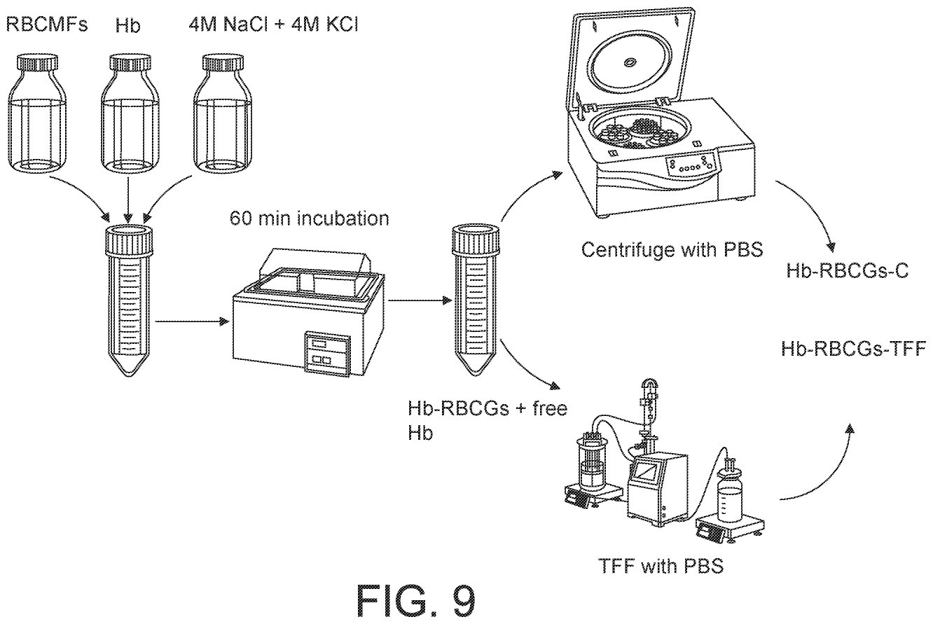

RBCG Resealing Process. Resealing RBCMFs to form resealed RBCGs was achieved by mixing a hypertonic solution with RBCMFs and purified Hb. Hb, purified via methods described in literature, was added to the concentrated RBCMFs produced above, and used at a Hb concentration of ~100 mg/mL. Aliquots of 4 M NaCl and 4 M KCl, taken from stock solutions, were added via syringe pump to a stirring mixture of RBCMFs and purified Hb to achieve Na⁺ and K⁺ concentrations of approximately 0.77 M and 0.021 M, respectively. These salt levels are significantly higher than physiological salt levels, thus creating a hypertonic environment. The mixture was cooled for 5 minutes at −20° C., and then incubated at 37° C. for 60 minutes. The resulting Hb-encapsulated resealed RBCGs (Hb-RBCGs) had their volume fraction adjusted to ~45% before being stored at 4° C. for further analysis. As a control to test various biophysical properties of the RBCGs, empty RBCGs were also prepared, without any encapsulated protein. The RBCMF resealing process used to encapsulate Hb to yield washed Hb-RBCGs is shown in FIG. 9 and shows both TFF and centrifugation modalities.

RBCG Wash Via Centrifugation. The first method explored for removing unencapsulated Hb from resealed Hb-RBCGs was differential centrifugation. This method is the industry standard for washing RBCs and other micron-scale particles. The applied centrifugal force ranges between 500-1,500 g for differential centrifugation of RBCs due to its relative lack of detrimental effects on the RBC lipid membrane. However, Hb-RBCGs required a slightly higher centrifugal force to properly separate the pelleted Hb-RBCGs from the supernatant, which may be due to their smaller size and relatively lower Hb content (i.e. density) than intact RBCs. Thus, Hb-RBCGs were washed by centrifuging the solution at 2,000 g for 20 minutes to form a pellet of Hb-RBCGs. After supernatant removal, the Hb-RBCG pellet was resuspended with fresh PBS. This process was repeated 3 times. After the final centrifugation step, the volume fraction of the Hb-RBCGs was adjusted via the addition of PBS to achieve ~45% v/v of Hb-RBCGs, to mimic a physiological RBC hematocrit. Hb-RBCGs purified via centrifugation are referred to as Hb-RBCGs-C.

RBCG Wash Via TFF. A bench-scale TFF module with a 500 kDa MWCO was used to remove unencapsulated Hb from resealed Hb-RBCGs, which has a molecular weight of ~64 kDa. The initial 50 mL sample of Hb-RBCGs was concentrated down to ~40 mL, before being subjected to constant volume diafiltration using PBS as the wash solution. The constant volume diafiltration process was terminated when the permeate concentration of cell-free Hb was below 0.25 mg/mL, at which point the cell-free Hb levels remaining in the Hb-RBCGs was negligible. At this end point, the washed Hb-RBCGs were concentrated to attain a volume fraction of ~45% Hb-RBCGs. The samples were stored in the refrigerator for further analysis. Hb-RBCGs purified via TFF are referred to as Hb-RBCGs-TFF.

ζ Potential Analysis. ζ potential analysis was performed on all fresh and thawed RBCMFs using a Brookhaven Instruments ZetaPals instrument (Holtsville, NY), and followed a procedure similar to Huang et al. RBCMFs were diluted 100× to a concentration of ~2×10⁷ RBCMFs/mL in deionized (DI) water for analysis.

Transmission Electron Microscopy (TEM) Analysis. The morphology of RBCs and RBCMFs was analyzed using a FEI Tecnai G2 Biotwin transmission electron microscope (TEM) (FEI, Hillsboro, OR). All samples were diluted 100× in PBS for analysis.

Coulter Counter Size Analysis. To analyze the size distribution of RBCMFs on the micron scale, Coulter Counter (CC) analysis was performed on a 100× dilution of the sample, which was further diluted by adding 0.2 mL of the diluted sample into 20 mL of double-filtered Isoton-III buffer (Beckman Coulter, Brea, CA). Particle counts and size distribution data were collected from each sample, and each sample was prepared identically. This method is preferable over dynamic light scattering (DLS) for analyzing particles in this size range, due to the difficulty that DLS often encounters when measuring non-spherical particles that are larger than 1 μm in diameter.

Quantification of Hb Concentration. All cell-free Hb concentrations were measured using the Winterbourn assay. Samples were diluted to <1 mg/mL Hb, and 1 mL was pipetted into a disposable cuvette. The absorbance spectrum of each sample was measured using an OLIS 8452A diode array spectrophotometer (OLIS, Athens, GA). Due to the fact that large particles scatter light, RBCMF retentate samples were additionally centrifuged and filtered through a 0.2 μm poly-ether sulfone filter. This was acceptable, since the assay aims to quantify the amount of cell-free Hb present in the solution and does not require the presence of the RBCMFs themselves.

Hb Retention in Hb-RBCGs After Washing. Hb encapsulation inside Hb-RBCGs was calculated by measuring the quantity of cell-free Hb removed from the Hb-RBCGs after the washing procedure relative to the initial bolus of Hb added. Cell-free Hb concentrations were measured using the Winterbourn assay using a method similar to that described above. Since the starting mass of Hb is known, the remaining Hb in the washed Hb-RBCGs must be encapsulated inside the RBCGs themselves. To account for slight variations in the initial concentration of RBCMFs used to prepare Hb-RBCGs due to the different batches of materials produced, Hb retention inside the washed Hb-RBCGs was normalized by dividing it by the final volume fraction occupied by the Hb-RBCGs. This yields a normalized Hb encapsulation efficiency.

Viscosity Analysis. The viscosity of Hb-RBCGs-C and Hb-RBCGs-TFF were compared to controls of unmodified RBCs and empty RBCGs at 45% volume fraction using a Brookfield-Ametek LV Rheometer (Middleborough, MA). 500 μL of sample was loaded into the rheometer, and the viscosity at a shear rate of 160 s⁻¹ was measured at 37° C.

RBCG Size Analysis. RBCG size distributions were measured via CC using the method described above. A 100× dilution of the sample was taken, which was further diluted by adding 0.2 mL of the diluted sample into 20 mL of double-filtered Isoton-III buffer (Beckman Coulter, Brea, CA). Particle counts and size distribution data were collected from each sample, and each sample was prepared identically.

Oxygen Equilibrium and Offloading Analysis. To assess any differences in the oxygen equilibria and oxygen offloading kinetics between Hb-RBCGs-TFF and Hb-RBCGs-C, the oxygen equilibrium curve (OEC) and the kinetics of oxygen offloading were measured using a Hemox Analyzer (TCS Scientific, New Hope, PA) and a stop-flow UV-visible spectrometer (Applied Photophysics Ltd., Surrey, UK), respectively. The OEC was measured and analyzed via the Hill equation to regress the partial pressure of oxygen (pO₂) where 50% of the Hb is saturated with oxygen (P₅₀) and the Hill coefficient (n). The OEC for Hb-RBCGs were compared against OECs for cell-free Hb and fresh RBCs. Oxygen offloading kinetics were measured and analyzed as described in the literature to regress the oxygen offloading rate constant (k_{off,O2}) for Hb-RBCGs and compared against values for cell-free Hb and RBCs.

Statistical Analysis. The IBM SPSS package (IBM, Armonk, NY) was used for all statistical analysis, and statistical significance was set at p<0.05. Except where otherwise noted, all tables report the mean value ±one standard deviation.

### Results and Discussion

In this example, we developed a simple and scalable method for preparing Hb-free RBCMFs from highly concentrated RBC solutions (30-40% volume fraction). TFF was used to remove cell-free Hb from lysed RBCs in the permeate stream, while retaining RBCMFs in the retentate. We also developed a scalable purification method to produce resealed RBCGs, that could be loaded with a therapeutic agent for systemic delivery. As our model encapsulant, we utilized Hb due to its ability to act as an oxygen therapeutic that can bind and release oxygen, and possible applications in further development of hemoglobin-based oxygen carriers. After resealing identically-prepared samples of Hb-containing RBCGs, centrifugation or tangential flow filtration was used to remove unencapsulated Hb from solution, leaving behind the Hb encapsulated RBCGs for subsequent biophysical analysis.

### RBCMF Size Measurements

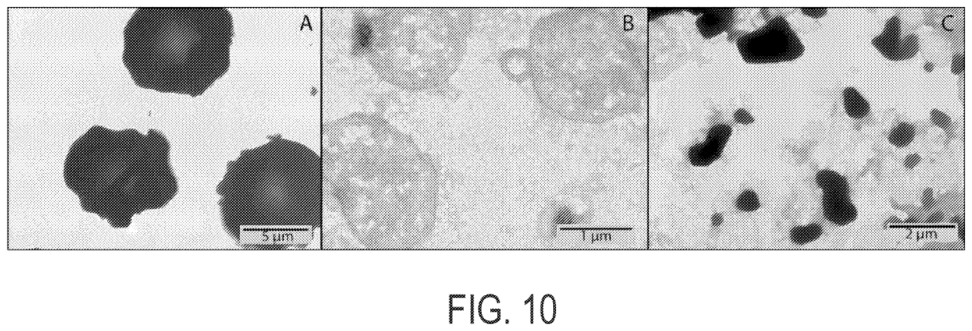

RBCMF Hydrodynamic Diameter and Morphology. Coulter Counter (CC) analysis of the unfiltered RBCMF species measured an average diameter of 2.70±0.051 μm. It is important to note that the hemolysis and subsequent TFF purification process exposes RBCMFs to shear stress, which most likely contributed to the existence of RBCMF species that fall within a smaller size distribution compared to intact RBCs, which were measured to have a diameter of 4.45±0.83 μm. The RBC diameter measured is smaller than reported values for RBCs in the literature, but consistent with measurements of RBCs using CC, due to the unique morphology of RBCs and the assumptions made by the CC machine. TEM image results are shown below in FIG. 10, and clearly indicate the morphological differences between RBCs, RBCMFs, and resealed Hb-RBCGs, respectively. RBCMFs (FIG. 10, panel B) roughly retain the overall rounded morphology of native RBCs (FIG. 10, panel A), while RBCGs have more irregular shapes. While the biconcave disk shape is optimal for RBC flow in capillaries, and since the RBCGs do not adhere to this morphology, their reduced size may help offset morphological changes, and other properties, such as solution viscosity, which will be important to consider when assessing their flow behavior in the microcirculation.

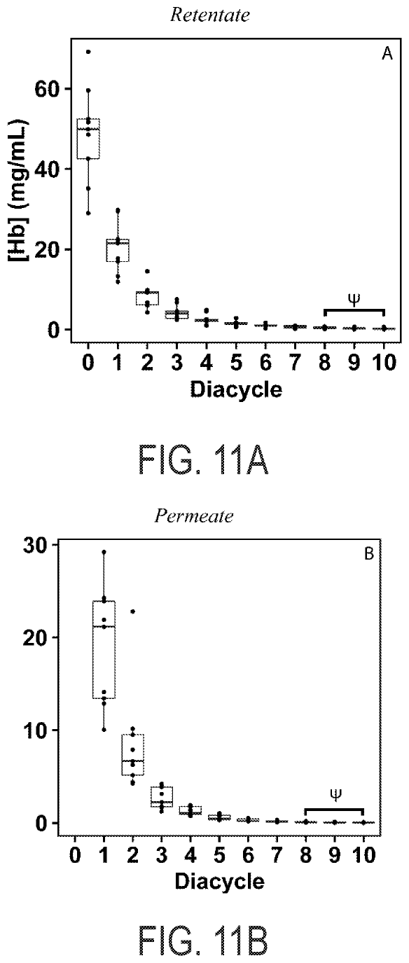

After RBC lysis and during TFF purification of RBCMFs, the concentration of cell-free Hb in the permeate decreased from an average of 19.0±2.2 mg/mL to 0.04±0.01 mg/mL after 10 diacycles. Similarly, the cell-free Hb concentration in the retentate decreased from 48.7±4.0 mg/mL to 0.3±0.07 mg/mL. FIGS. 11A-11B show the reduction in cell-free Hb with increasing diacycles. Both reductions (i.e., permeate and retentate) in cell-free Hb levels were statistically significant (p<0.05) when comparing initial and final cell-free Hb concentrations to each other. The RBCMF purification protocol demonstrates the ability to remove >99% of the cell-free Hb present in the initially lysed RBC solution. The 0.65 μm HF membrane was effective in removing cell-free Hb and other debris below its MWCO, while retaining RBCMFs larger than the MWCO. Similar findings were observed for all replicates (N=10). Differences in cell-free Hb retentate concentrations were significant (p<0.05) until after the eighth diacycle, where subsequent diacycles failed to yield significant changes in the cell-free Hb concentration. The cell-free Hb differences in the permeate were significantly different until after the seventh diacycle, where again subsequent diacycles failed to yield significant reduction in the cell-free Hb concentration.

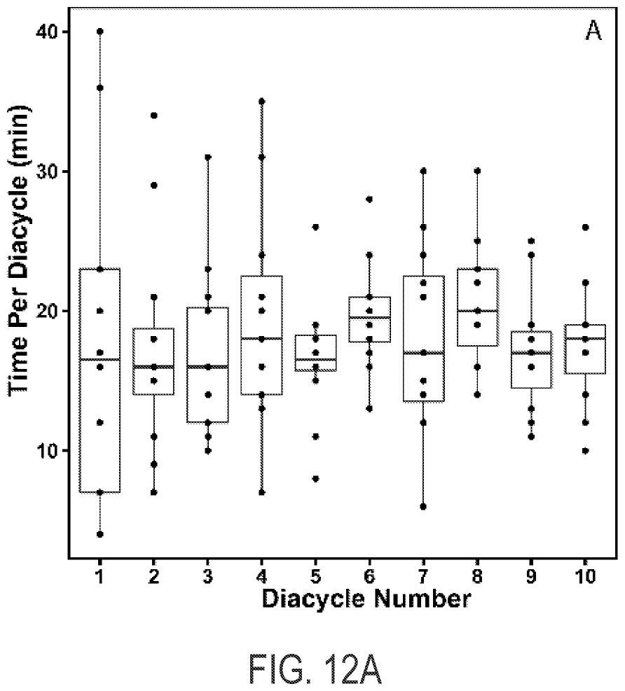

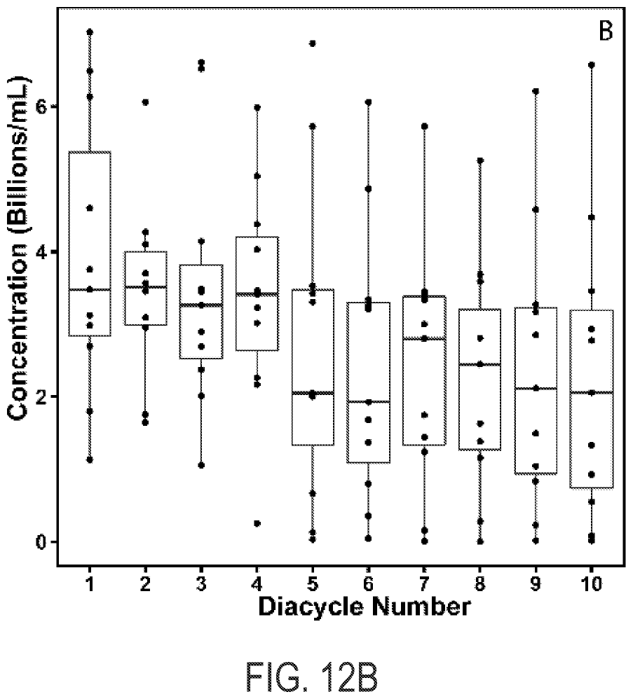

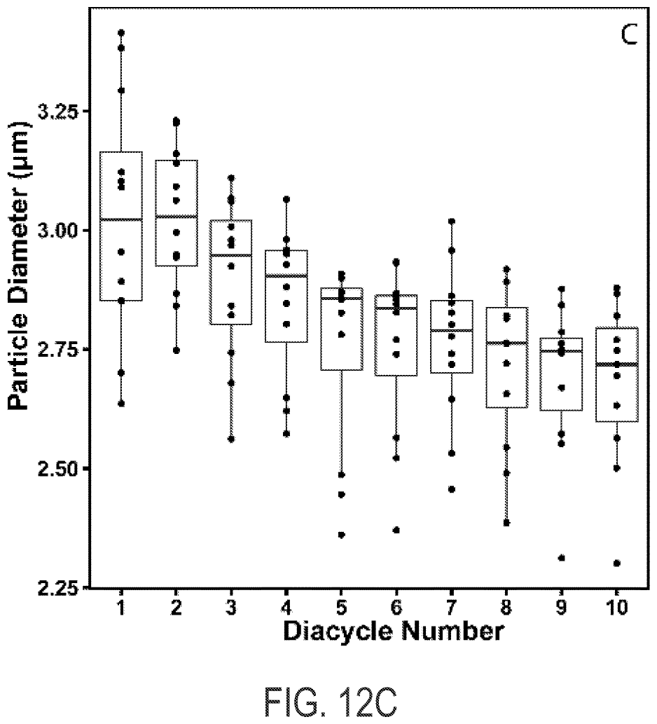

Effect of TFF on Diacycle Time and RBCMF Concentration and Size. Analysis of the individual TFF diacycles required to remove >99% Hb yielded insights into the effects of the TFF washing process on the diacycle time and RBCMF concentration and size, and these findings are summarized in FIGS. 12A-12C, with n=11 replicates. As shown in FIG. 12A, the time required to complete each diacycle did not significantly increase with increasing number of diacycles (p >0.05). Outside factors such as the number of times each TFF cartridge had been previously used had an impact on batch-to-batch diacycle times, but overall no significant change in diacycle time was observed, both within each replicate and when combining all replicates together. Additionally, no significant change in RBCMF concentration was observed with increasing number of diacycles (p >0.05), and particle size was only slightly impacted. Specifically, diacycles 1 and 2 had significantly larger RBCMFs than diacycles 6-10 (p<0.05), though no other significant size differences across any of the groups were detected. FIG. 12B and FIG. 12C highlight these trends, and suggest that the shear forces applied in the TFF circuit do not significantly alter the concentration and size of RBCMFs for the 10 diacycles required to remove 99% of the cell-free Hb. The lack of change in RBCMF concentration and only slight decrease in particle size suggest that TFF can acceptably perform its major function of removing cell-free Hb without significantly altering the size of the resulting RBCMFs.

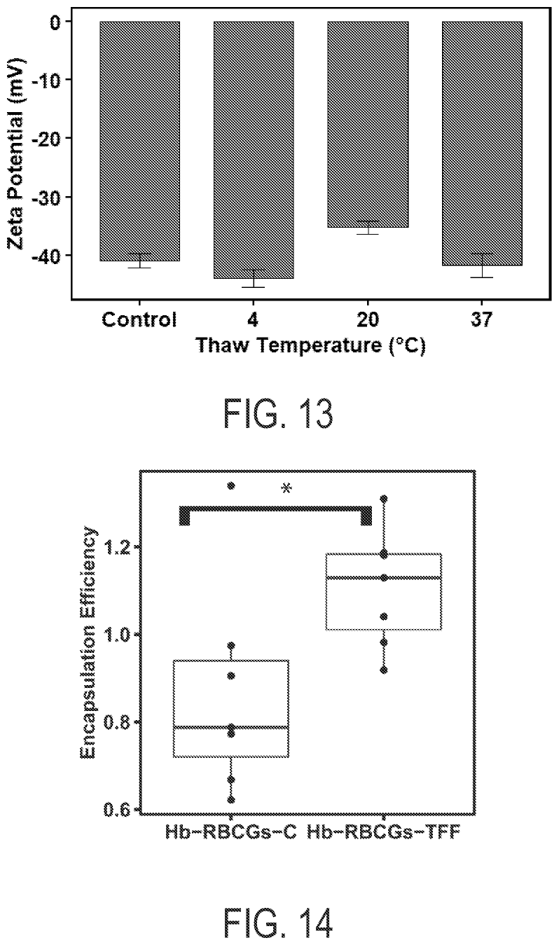

Effect of Freeze-Thaw Cycle on the Zeta Potential of RBCMFs Indicates Their Storage Stability. Data for zeta potential analysis was reported as the mean±one standard error. The ζ potential of fresh RBCMFs was −40.8±1.2 mV. Thawing frozen RBCMFs at 4° C. lowered the ζ potential of RBCMFs to −43.8±1.5 mV. Thawing frozen RBCMFs at 20° C. raised the ζ potential of the RBCMFs to −35.2±1.1 mV, while thawing frozen RBCMFs at 37° C. results in a ζ potential of −41.6±2.0 mV. FIG. 13 summarizes these changes. However, no significant differences in ζ potential were observed between groups as a result of freeze-thawing. Therefore, subjecting RBCMFs to a single freeze-thaw cycle did not significantly alter the ζ potential when compared to control values, regardless of thaw temperature. This is a promising result, as it suggests that the RBCMF precursors required to form RBCGs may be stored and thawed under conditions that would normally destroy RBCs i.e. being frozen without any cryoprotectant. This would allow for large batches of precursor RBCMFs to be produced, stored in a freezer, and then thawed out as needed to manufacture RBCGs.

These ζ potentials are more negative than reported ranges of −9.3 to −15 mV for fresh RBCs, but are consistent with observations that the ζ potential is more negative for RBCMFs than intact RBCs. After hemolysis, cell-free Hb, which is a tetramer containing four iron (II) atoms each, is removed from both inside the RBCMF as well as from the extracellular space, which can explain the reduction in ζ potential of RBCMFs.

TFF of Resealed Hb-RBCGs Results in Higher Hb Encapsulation Compared to Centrifugation, and Places an Upper Bound on an Acceptable Number of Diacycles. RBCGs encapsulating cargo must be washed in order to remove unencapsulated material and is a necessary processing step for many biomedical applications. Ideally the washing method should minimize damage to the resealed RBCGs in order to maximize the yield of resealed RBCGs encapsulating cargo. In this study, Hb was used as the model encapsulant, because of the availability of assays to measure Hb activity in our lab. Hb was encapsulated inside RBCGs and subsequently washed via TFF or differential centrifugation to remove unencapsulated protein. When normalizing the Hb encapsulation efficiency (i.e. fraction of encapsulated Hb compared to the initial Hb basis) to the volume fraction occupied by the resealed RBCGs themselves in solution (i.e. equivalent to the hematocrit), Hb-RBCGs-TFF retained a significantly higher amount of Hb compared to Hb-RBCGs-C (p<0.05) prepared from the same batch of precursor RBCMFs. TFF has been used to wash RBCs. In this current application of TFF for particle washing, Hb-RBCGs were much smaller in size compared to their precursor RBCs and RBCMFs, which may lead to a difference in the centrifugal force being required to successfully pellet out the Hb-RBCGs. FIG. 14 highlights this difference, with Hb-RBCGs-C possessing a normalized Hb encapsulation efficiency of 0.87±0.091 versus 1.10±0.051 for Hb-RBCGs-TFF (p<0.05). This normalization was employed to facilitate easy comparison between replicates, to account for batch-to-batch differences in the initial concentration of RBCMFs used to manufacture the resealed RBCGs. While Hb has been known to reversibly bind to the RBC membrane, we have demonstrated from the studies above that TFF is sufficient to remove over 99% of the cell-free Hb from RBCMFs after 10 diacycles. The observed lysis of Hb-RBCGs and higher loss of Hb present in Hb-RBCGs-C compared to Hb-RBCGs-TFF suggests that centrifugation is more damaging to Hb-RBCGs versus TFF. Furthermore, TFF is more amenable to scale up versus centrifugation, which makes TFF more appealing for larger-scale applications where a higher volume of RBCGs may be needed. While this work employed 10 diacycles for the Hb-RBCG TFF washing protocol, an increase in the concentration of cell-free Hb was observed after the 7^{th} diacycle for various replicates. This phenomenon is believed to be similar to the hemolysis encountered by RBCs after prolonged exposure to TFF, and thus the authors recommend that future work with this method reduce the total number of diacycles from 10 to 7 to reduce leakage of encapsulated material from the RBCGs. The resealed Hb-RBCGs, which are prone to shear-induced hemolysis, should not be subjected to more diacycles than is necessary.

Characterization of Hb-RBCG Physical Properties. In this Example, the particle concentration, diameter and viscosity of replicate RBCGs were measured and compared against native RBCs, RBCMFs, and Hb. Table 1 summarizes these data, and from it several important details become apparent. With n=7 replicates, there was no significant difference observed between the viscosity of RBCs and any of the RBCG samples (p >0.05). RBCs were significantly larger in diameter compared to RBCMFs (p<0.05) and Hb-RBCGs-TFF (p<0.05), with a mean diameter of 4.45±0.83 μm. Hb-RBCGs-C had a very wide size distribution, with a mean diameter of 9.65±4.07 μm. In contrast, Hb-RBCGs-TFF possessed a mean diameter of 3.02±1.21 μm. When measuring the size distribution of Hb-RBCGs-C via CC, we observed particle diameters near the upper detection limit of 18 μm for the specific CC aperture used for this analysis, as well as a very wide distribution that exceeded the size of RBCs. This suggested extreme particle aggregation. Further experiments were done to assess the aggregation observed and correlate it with the centrifugation process, the results of which are summarized in Section 3.8. Furthermore, while no statistically significant difference was observed between the viscosities of the samples, centrifuged RBCGs exhibited a viscosity that was almost double that of the other samples, as shown in Table 4. This, again, raised concerns of RBCG aggregation resulting in larger particles, which could increase the solution viscosity, and could be potentially hazardous to patient health.

TABLE 4
Biophysical properties of RBCGs. The viscosity, mean diameter,
and concentration of RBCGs, cell-free Hb, RBCMFs, and fresh
RBCs are reported. For viscosity measurements, all samples
except Hb had their volume fraction adjusted to 45% to mimic
physiological RBC concentrations in the blood.

| | Concentration | Mean Diameter | Viscosity |
|---|---|---|---|
| Sample | (10⁹ particles/mL) | (μm) | (cP) |
| Hb-RBCGs-C | 4.37 ± 1.63 | 8.92 ± 4.03 | 7.08 ± 1.31 |
| Hb-RBCGs-TFF | 48.4 ± 12.45 | 3.09 ± 1.57 | 4.48 ± 0.37 |
| Cell-Free Hb | Variable | 0.005 | Concentration- |
| | | | dependent |
| RBCs | 4.63 ± 0.35 | 4.74 ± 0.76 | 3.93 ± 1.77 |
| RBCMFs | 2.70 ± 0.176 | 2.68 ± 0.17 | 4.53 ± 1.27 |

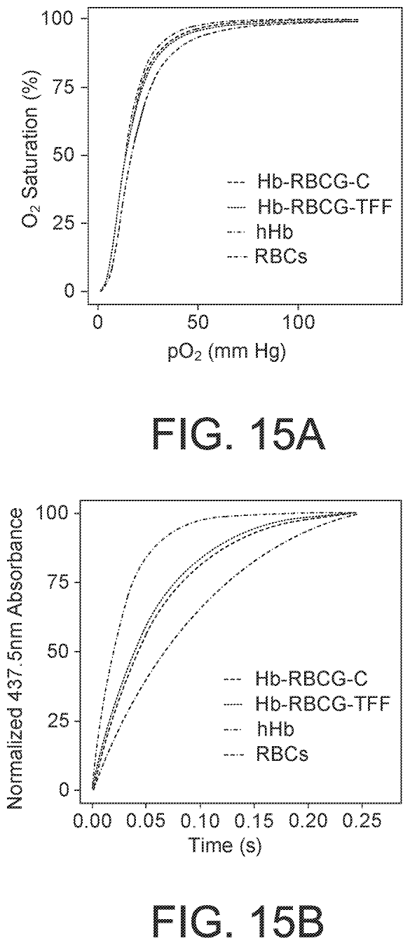

Oxygen-Binding Equilibria and Oxygen Offloading Kinetics of Hb-RBCGs. It was important to determine whether the oxygen-binding equilibria and oxygen offloading kinetics of Hb-RBCGs were being altered by the Hb-RBCG washing process, and those properties are summarized in Table 2 as well as FIGS. 15A-15B. Typical data for fresh RBCs and Hb were sourced from Banerjee et al. Neither the P₅₀, cooperativity coefficient (n), nor k_{off,O2}, were significantly different between Hb-RBCGs-TFF and Hb-RBCGs-C (p<0.05). Both types of Hb-RBCGs had a lower P₅₀ than fresh RBCs (p<0.05), and a higher P₅₀ than cell-free Hb (p<0.05). In addition, k_{off,O2} for both types of Hb-RBCGs were significantly higher than RBCs (p<0.05) and lower than cell-free Hb (p<0.05). For the Hb-RBCGs-TFF, these changes can largely be attributed to the size differences between the Hb-RBCGs-TFF and either native RBCs or cell-free Hb. The path length for oxygen diffusion in RBCs is much longer than Hb-RBCGs and cell-free Hb due to their larger diameter. For Hb-RBCGs-C, while the mean particle size was larger than RBCs, the wide size distribution of particles present suggest that there were particles smaller than RBCs. The presence of these smaller particles would skew measurements of the oxygen equilibrium and oxygen offloading kinetics towards those for Hb-RBCGs-TFF.

TABLE 5
Oxygen release kinetics and equilibria of Hb-RBCGs compared to
cell-free Hb and fresh RBCs. The first-order rate constant for oxygen
offloading is reported, along with the P₅₀ and cooperativity
coefficient regressed from the oxygen equilibrium curve.

| | P₅₀ (mm Hg) | n | k_{off, O2} (s⁻¹) |
|---|---|---|---|
| Hb-RBCGs-C | 13.90 ± 0.32 | 2.37 ± 0.04 | 13.05 ± 1.75 |
| Hb-RBCGs-TFF | 14.22 ± 0.19 | 2.393 ± 0.04 | 16.68 ± 0.89 |
| Cell-free Hb | 12.55 ± 0.97 | 2.64 ± 0.09 | 36.51 ± 2.53 |
| RBCs | 27.16 ± 4.45 | 2.21 ± 0.13 | 8.61 ± 2.55 |

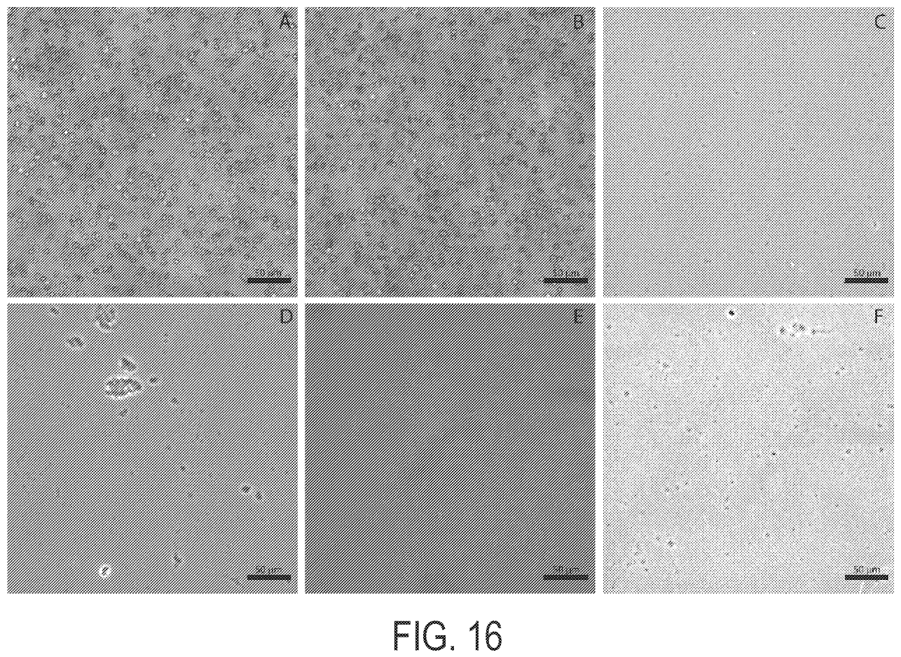

Centrifugation Elicits Deleterious Aggregation of RBCGs. As discussed above, we observed that CC measurements of Hb-RBCGs-C, while yielding a mean particle diameter that was measurable, also counted some particles near the upper detection limit of the installed CC column. In addition, the concentration of centrifuged RBCGs was lower than expected, since the final volume of RBCGs was lower than that of the precursor RBCs. These observations, compounded with the rapid and severe pelleting observed for the Hb-RBCGs-C but not the Hb-RBCGs-TFF, prompted the utilization of confocal and phase-contrast microscopy to validate the higher observed colloidal stability of the particles. All stages of the RBCG manufacturing process were imaged and shown in FIG. 16. Most notably, Hb-RBCGs-C (FIG. 16, panel D) exhibited signs of heavy particle aggregation, with some particles measuring almost 50 μm. Empty RBCGs, which were centrifuged once to standardize the volume fraction to 45%, showed aggregation to a lesser extent, as shown in FIG. 16, panel E, although they were difficult to image and required high contrast due to not containing Hb. Some of these particles are much larger than the detection limit of the CC instrument, therefore it is likely that these particles were not properly being measured. Attempts to mitigate particle aggregation via thorough mixing and sonication were unsuccessful, as the force required to separate the aggregates also induced lysis and release of the encapsulated Hb, further lowering the yield of Hb-RBCGs-C. In contrast, Hb-RBCGs-TFF as shown in FIG. 16, panel F do not show this extreme aggregation, which suggests that this method is much less likely to create Hb-RBCG aggregates.

### CONCLUSION

We demonstrate that TFF is a scalable process for the production of purified RBCMFs. Potential adverse effects due to the presence of cell-free Hb should be mitigated by the removal of >99% of the cell-free Hb present in the RBCMF preparations via TFF. The resealing of TFF-purified RBCMFs in the presence of the model protein Hb via ionic reversal was shown to be a viable approach for the production of Hb-RBCGs. Upon formation of Hb-RBCGs, we demonstrated the ability of TFF remove unencapsulated Hb, and observed that TFF-washed Hb-RBCGs possessed a higher protein encapsulation efficiency and much lower degree of aggregation than Hb-RBCGs washed via centrifugation.

The compositions and methods of the appended claims are not limited in scope by the specific compositions and methods described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A method for forming a composition of purified red blood cell membrane fragments (RBCMFs), the method comprising:
lysing a population of red blood cells (RBCs) to produce a composition comprising crude RBCMFs; and
filtering the crude RBCMFs by microfiltration against a filtration membrane, thereby forming a retentate fraction comprising the purified RBCMFs and a permeate fraction comprising low molecular weight contaminants.

2. The method of claim 1, wherein the purified RBCMFs comprise less than 1% by weight free hemoglobin and free heme, such as less than 0.5% by weight free hemoglobin and free heme, or less than 0.1% by weight free hemoglobin and free heme.

3. The method of any of claims 1-2, wherein the filtration membrane is rated for a 0.65 μm molecular weight cut-off.

4. The method of any of claims 1-3, wherein filtering the crude RBCMFs by microfiltration comprises filtering for at least 3 diafiltrations cycles, such as at least 4 diafiltration cycles, at least 5 diafiltration cycles, at least 6 diafiltration cycles, at least 7 diafiltrations cycle, at least 8 diafiltration cycles, at least 9 diafiltrations cycle, or at least 10 diafiltration cycles.

5. The method of any one of claims 1-4, wherein the microfiltration comprises tangential flow filtration.

6. The method of any of claims 1-5, wherein the method further comprises washing a population of red blood cells by microfiltration against a filtration membrane prior to the lysing step, thereby forming a retentate fraction comprising a population of washed red blood cells and a permeate fraction comprising cell-free hemoglobin and other low molecular weight contaminants.

7. The method of claim 6, wherein the washed red blood cells comprise less than 1% by weight free hemoglobin and free heme, such as less than 0.5% by weight free hemoglobin and free heme, or less than 0.1% by weight free hemoglobin and free heme.

8. The method of any of claims 6-7, wherein the filtration membrane is rated for a 0.65 μm molecular weight cut-off.

9. The method of any of claims 6-8, wherein washing the population of red blood cells by microfiltration comprises filtering for at least 3 diafiltrations cycles, such as at least 4 diafiltration cycles, at least 5 diafiltration cycles, at least 6 diafiltration cycles, at least 7 diafiltrations cycle, at least 8 diafiltration cycles, at least 9 diafiltrations cycle, or at least 10 diafiltration cycles.

10. The method of any one of claims 1-9, wherein the microfiltration comprises tangential flow filtration.

11. The method of any of claims 1-10, further comprising modifying the size of the purified RBCMFs.

12. The method of claim 11, wherein modifying the size of the purified RBCMFs comprises sonicating a composition comprising the purified RBCMFs to form a population of size-reduced RBCMFs.

13. The method of claim 11, wherein modifying the size of the purified RBCMFs comprises homogenizing a composition comprising the purified RBCMFs to form a population of size-reduced RBCMFs.

14. The method of claim 11, wherein modifying the size of the purified RBCMFs comprises shearing a composition comprising the purified RBCMFs to form a population of size-reduced RBCMFs.

15. The method of claim 11, wherein modifying the size of the purified RBCMFs comprises extruding a composition comprising the purified RBCMFs to form a population of size-reduced RBCMFs.

16. The method of any of claims 11-15, wherein the population of size-reduced RBCMFs exhibit an average particle size of from 10 nm to 200 nm, as measured by dynamic light scattering (DLS).

17. The method of any of claims 1-16, further comprising encapsulating one or more active agent(s) in the purified RBCMFs or the size-reduced RBCMFs.

18. The method of claim 17, wherein encapsulating the active agent(s) in the purified RBCMFs or the size-reduced RBCMFs comprises contacting the purified RBCMFs or the size-reduced RBCMFs with the active agent(s) followed by ionic reversal to reseal the purified RBCMFs or the size-reduced RBCMFs.

19. The method of claim 17, wherein encapsulating the active agent(s) in the purified RBCMFs or the size-reduced RBCMFs comprises ionic reversal to reseal the purified RBCMFs or the size-reduced RBCMFs followed by electroporation to induce uptake of the active agent(s).

20. The method of any of claims 17-19, further comprising removing the unencapsulated active agent(s) from the active agent(s) encapsulated in the resealed RBCMFs or the resealed size-reduced RBCMFs via microfiltration or ultrafiltration.

21. The method of claim 20, wherein the filtration comprises tangential flow filtration.

22. The method of any of claims 1-21, further comprising functionalizing a particle with the purified RBCMFs or the size-reduced RBCMFs.

23. The method of any one of claims 1-22, further comprising conjugating one or more polymers to the purified or resealed RBCMFs or the purified or resealed size-reduced RBCMFs.

24. The method of claim 23, wherein the polymer comprises a hydrophilic polymer or a zwitterionic polymer.

25. The method of any of claims 23-24, wherein the polymer comprises a poly(alkylene oxide), such as polyethylene glycol (PEG).

26. The method of any of claims 1-25, wherein the red blood cells comprise human red blood cells.

27. The method of any of claims 1-25, wherein the red blood cells comprise non-human red blood cells.

28. The method of any of claims 1-27, wherein the red blood cells comprise red blood cells derived from stem cells, “recombinant red cells” cultured in bioreactors, or engineered red cells obtained from genetic engineering of stem cells.

29. A composition comprising red blood cell ghosts (i.e., resealed RBCMFs or resealed size-reduced RBCMFs) prepared by the method of any of claims 1-28.

30. A composition comprising a population of particles functionalized with RBCMFs or size-reduced RBCMFs prepared by the method of any of claims 1-28.

31. The use of the composition of any of claims 29-30 for drug delivery, diagnostics, or combination of the two.
